(19) United States
(12) Patent Application Publication
(10) Pub. No.: US 2025/0104450 A1
(43) Pub. Date: Mar. 27, 2025

(54) SYSTEMS AND METHODS FOR PREDICTING SLIDE-LEVEL CLASS LABELS FOR A WHOLE-SLIDE IMAGE

(71) Applicant: Foundation Medicine, Inc., Boston, MA (US)

(72) Inventor: James PAO, Boston, MA (US)

(73) Assignee: Foundation Medicine, Inc., Boston, MA (US)

(21) Appl. No.: 18/910,993

(22) Filed: Oct. 9, 2024

Related U.S. Application Data
(63) Continuation of application No. PCT/US2023/018074, filed on Apr. 10, 2023.
(60) Provisional application No. 63/329,730, filed on Apr. 11, 2022.

(51) Int. Cl. G06V 20/69; G06T 7/00; G06T 7/11; G06V 10/771; G16H 20/00; G16H 30/40

(52) U.S. Cl. CPC G06V 20/698; G06T 7/0012; G06T 7/11; G06V 10/771; G16H 20/00; G16H 30/40; G06T 2207/20084; G06T 2207/30024

(57) ABSTRACT

A method implemented by one or more processors includes segmenting an image into a plurality of patches grouping the plurality of patches into at least one bag of patches, and inputting the at least one bag of patches into a machine-learning model trained to generate a prediction of an image class label based on the at least one bag of patches. The machine-learning model includes a first layer trained to generate one or more feature maps based on the at least one bag of patches, a second layer trained to normalize the one or more feature maps utilizing a set of batch normalization parameters determined from the at least one bag of patches to generate one or more normalized feature maps, and a third layer trained to generate the prediction of the image class label based at least in part on the one or more normalized feature maps.

### CROSS-REFERENCE TO RELATED APPLICATIONS

This application is a continuation application of International Application No. PCT/US2023/018074, filed internationally on Apr. 10, 2023, which claims the benefit of U.S. Provisional Application No. 63/329,730 filed Apr. 11, 2022, each of which is hereby incorporated by reference in its entirety.

### TECHNICAL FIELD

This application relates generally to whole-slide images, and, more particularly, to predicting slide-level class labels for whole-slide images.

### BACKGROUND

Multiple-instance learning (MIL) is a machine-learning technique in which a MIL machine-learning model trains on inputs of sets of image instances (e.g., referred to as a “bag” of patches of pixels) as opposed to the individual image instances themselves. For example, a bag of patches of pixels may be structured and pre-processed in a manner in which ground truth class labels are assigned to the bags of patches of pixels for the purposes of training the MIL model. For example, a histopathology image, such as a hemotoxin and eosin (H&E) slide, may include a tissue sample that may be further sequenced and analyzed. In such an instance, one or more biomarkers or gene alterations may be determined as being associated with the whole tissue sample as opposed to, for example, any specific tissue cells within the tissue sample. Thus, the MIL model may be particularly suitable for analyzing and classifying bags of patches of pixels in histopathology images, which may often include very large and high-resolution images. However, because the bags of patches of pixels are each taken from the same histopathology image, traditional machine-learning model techniques of tracking data, e.g., statistics based on the data, as the model trains on batches of varying images may be unsuitable, and may further diminish the overall predict performance of the MIL model. It may be useful to provide techniques to improve MIL models for predicting slide-level class labels for a singular whole-slide image.

### INCORPORATION BY REFERENCE

The disclosures of all publications, patents, and patent applications referred to herein are each hereby incorporated by reference in their entireties. To the extent that any reference incorporated by reference conflicts with the instant disclosure, the instant disclosure shall control.

### SUMMARY

Embodiments of the present disclosure are directed toward one or more computing devices, methods, and non-transitory computer-readable media that may generate inference-phase-specific batch normalization parameters for a machine-learning model trained to predict a slide-level class label describing one or more gene alterations or other biomarkers based on a singular whole-slide histopathology image. For example, a multiple-instance learning convolutional neural network (MILCNN) may include, for example, one or more convolutional layers, one or more pooling or max-pooling layers, one or more fully-connected layers, one or more batch normalization layers, and one or more rectified linear units (ReLUs). During a training phase of the MILCNN, the one or more batch normalization layers may utilize one or more sets of mini-batch normalization parameters (e.g., a mini-mean parameter and a mini-variance parameter) to normalize features in a feature map at each layer of the MILCNN.

Specifically, during the training phase of the MILCNN, each batch of data is normalized by subtracting the batch mean and dividing by the square root of the batch variance. However, during an inference phase of the MILCNN, at least one bag of patches of pixels of a singular whole-slide histopathology image may be inputted to the MILCNN and the MILCNN may predict a slide-level label (e.g., predict a slide-level label based on the entire bag of patches of pixels as opposed to the individual patches of pixels constituting the bag). Thus, during the inference phase, the MILCNN may generate and utilize inference-phase-specific batch normalization parameters, such that the one or more batch normalization layers may normalize features in a feature map at each feature layer of the MILCNN utilizing the inference-phase-specific batch normalization parameters as opposed to utilizing, for example, a running mean and variance calculated based on the one or more sets of mini-batch normalization parameters (e.g., a mini-mean parameter and a mini-variance parameter) learned during the training phase of the MILCNN.

By generating inference-phase-specific batch normalization statistics for the MILCNN, the trained MILCNN may better predict slide-level class labels describing one or more gene alterations or other biomarkers (e.g., an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, neurotrophic tyrosine receptor kinase (NTRK) genes 1/2/3, and so forth) based on a singular whole slide histopathology image. Particularly, in accordance with the presently-disclosed embodiments, by generating inference-phase-specific batch normalization parameters, the trained MILCNN may include batch normalization parameters that are appropriately “fitted” to the data, e.g., inference data or input data, which includes bags of patches of pixels all corresponding to a singular whole-slide histopathology image (e.g., as opposed to being overly “fitted” to only the most recent images inputted to the MIL model as would otherwise be the case utilizing training-phase-determined running mean and running variance batch normalization parameters).

In certain embodiments, during an inference phase of the machine-learning model, one or more computing devices, methods, and non-transitory computer-readable media may segment an image into a plurality of patches. For example, in one embodiment, the image includes only one whole-slide image (WSI). In some embodiments, the image may include an image of a tissue sample, and each patch of the plurality of patches may include a plurality of pixels corresponding to one or more regions of the image. In some embodiments, the image may include a histological stain image, a fluorescence in situ hybridization (FISH) image, an immunofluorescence (IF) image, or a hematoxylin and eosin (H&E) image.

In certain embodiments, the one or more computing devices may then group the plurality of patches into at least one bag of patches and input the at least one bag of patches into a machine-learning model trained to generate a prediction of an image class label based on the at least one bag of patches. In certain embodiments, the machine-learning model may include a first layer trained to generate one or more feature maps based on the at least one bag of patches, a second layer trained to normalize the one or more feature maps utilizing a set of batch normalization parameters determined from the at least one bag of patches to generate one or more normalized feature maps, and a third layer trained to generate the prediction of the image class label based at least in part on the one or more normalized feature maps. For example, in one embodiment, the machine-learning model may include one or more convolutional neural networks (CNNs). In another embodiment, the machine-learning model may include one or more fully connected neural networks (FCNNs). In some embodiments, the machine-learning model may include a multiple-instance learning (MIL) machine-learning model. In certain embodiments, the set of batch normalization parameters may include a mean and a variance determined from the at least one bag of patches. In some embodiments, the set of batch normalization parameters corresponds to only the at least one second bag of patches.

In certain embodiments, during a training phase of the machine-learning model, the one or more computing devices, methods, and non-transitory computer-readable media may receive a training image, segment the training image into a second plurality of patches, group the second plurality of patches into at least one second bag of patches, and input the at least one second bag of patches into the machine-learning model to generate a prediction of a second image class label based on the at least one second bag of patches. In certain embodiments, the first layer of the machine-learning may be trained to generate one or more feature maps based on the at least one second bag of patches. In certain embodiments, the second layer of the machine-learning model may be trained to normalize the one or more second feature maps utilizing a set of mini-batch normalization parameters determined from the at least one second bag of patches to generate one or more second normalized feature maps. In certain embodiments, the third layer of the machine-learning model may be trained to generate the prediction of the second image class label for the training image based at least in part on the one or more second normalized feature maps. For example, in some embodiments, the first layer may include one or more convolutional layers, the second layer may include one or more batch normalization layers, and the third layer may include an output layer.

In certain embodiments, the one or more batch normalization layers may be trained to compute at least one of a running mean, a running variance, a gamma parameter (i.e., a scaling parameter), and a beta parameter (i.e., an offset parameter) of each of a plurality of sets of mini-batch normalization parameters during the training phase of the machine-learning model. In some embodiments, in response to the machine-learning model being trained, one or more of the gamma parameter (i.e., scaling parameter) and the beta parameter (i.e., offset parameter) may be fixed. In some embodiments, in response to the machine-learning model being trained, one or more of a mean and a variance determined for the at least one second bag of patches is configured to be determined for each additional bag of patches from the at least one second bag of patches. For example, in some embodiments, the set of mini-batch normalization parameters may include a mini-batch mean and a mini-batch variance. In some embodiments, segmenting the training image into at least one second bag of patches may include randomly sampling one or more patches of pixels of the at least one second bag of patches.

In certain embodiments, the image class label may include an indication of a genetic biomarker of a tissue sample captured in the image. For example, in some embodiments, the genetic biomarker of the tissue sample may include an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, or a neurotrophic tyrosine receptor kinase (NTRK) genes 1/2/3. In certain embodiments, the one or more computing devices, methods, and non-transitory computer-readable media may then generate a report based on the prediction of the image class label indicating the genetic biomarker of the tissue sample. For example, the one or more computing devices, methods, and non-transitory computer-readable media cause one or more electronic devices to display the report, in which the one or more electronic devices includes a human machine interface (HMI) associated with a pathologist to display the report.

### BRIEF DESCRIPTION OF THE DRAWINGS

FIG. 1 illustrates an exemplary workflow diagram of a training phase for training a machine-learning model to predict a slide-level class label describing one or more gene alterations or other biomarkers based on a singular whole-slide histopathology image, according to some embodiments.

FIG. 2 illustrates an exemplary workflow diagram of an inference phase for utilizing a machine-learning model trained to predict a slide-level class label describing one or more gene alterations or other biomarkers based on a singular whole-slide histopathology image, according to some embodiments.

FIG. 3A illustrates an exemplary training phase and FIG. 3B illustrates an exemplary inference phase of a multiple-instance learning (MIL) convolutional neural network (CNN) model trained to predict a slide-level class label describing one or more gene alterations or other biomarkers based on a singular whole-slide histopathology image, according to some embodiments.

FIG. 4 illustrates a flow diagram of an exemplary method for utilizing a machine-learning model train to predict a slide-level class label describing one or more gene alterations or other biomarkers based on a singular whole-slide histopathology image, according to some embodiments.

FIG. 5 illustrates a flow diagram of an exemplary method for training a machine-learning model to predict a slide-level class label describing one or more gene alterations or other biomarkers based on a singular whole-slide histopathology image, according to some embodiments.

FIG. 6 illustrates an example computing system, according to some embodiments.

FIG. 7 illustrates a diagram of an example artificial intelligence (AI) architecture included as part of the example computing system of FIG. 6, according to some embodiments.

FIG. 8A shows a comparison of several exemplary deep learning models for predicting EGFR status from H&E images, with the MIL model being significantly better than comparator two-stage patch models and a weakly-supervised patch prediction model, according to some embodiments.

FIG. 8B shows cross-validated receiver operator curve for the MIL model, according to some embodiments.

FIG. 9A shows attention weights for EGFR prediction as separated by predicted tissue morphology, with all patches from 100 high-confidence bags (50 from EGFR mutant slides and 50 from wild-type slides), according to some embodiments. For both mutant and wild-type slides, the tumor patches received the most attention from the MIL model.

FIG. 9B shows median attention weight per tissue-morphology group for the 100 slides, according to some embodiments. Both mutant and wild-type slides had an appreciable median attention weight for tumor patches. Mutant slides had higher distribution of median patch attention for tumor and stroma patches than wild-type, whereas wild-type slides had higher distribution of median patch attention for immune, normal, and necrosis.

FIG. 9C shows maximum attention weight per tissue-morphology group for each of the 100 slides, according to some embodiments.

FIG. 9D shows EGFR tumor positive (TP) attention weights from bag of 250 patches, according to some embodiments. High attention given to tumor and stroma patches. (I-V) are acinar predominant pattern and hobnail cytology, with low peritumoral and intratumoral immune fractions, ranging 0.1-0.2. (IV) has a low presence of necrotic tissue (VI) was predicted as stroma by the tissue-morphology model, and pathologists confirmed this patch as fibrosis.

FIG. 9E shows EGFR tumor negative (TN) attention weights from bag of 250 patches, according to some embodiments. High attention given to tumor patches and some immune patches. (I-II) are acinar/lepidic pattern with hobnail cytology and intratumoral lymphoid aggregates. (III-VI) were predicted as tumor or immune foci by the tissue-morphology model. Pathologists confirmed high peritumoral and intratumoral immune fraction, ranging from 0.2-0.7, for these patches. Inflammation was noticeable present as well (IV).

FIG. 10A shows predominant architectural pattern of high-attention patches, determined by patch mode, by predicted status, for 49 pathologist reviewed bags, according to some embodiments. Bags with solid predominant architecture were significantly more likely to be predicted wild-type (p=0.032). High enrichment for mutant prediction for lepidic and papillary architectures.

FIG. 10B shows minor architectural pattern of high-attention patches, with strong enrichment for mutant status prediction for lepidic and micropapillary, for 49 pathologist reviewed bags, according to some embodiments.

FIG. 10C shows cytology for high-attention patches, determined by patch mode, with enrichment of mutant predictions for hobnail and columnar types and enrichment of wild-type predictions for mucinous and sarcomatoid types, for 49 pathologist reviewed bags, according to some embodiments.

FIG. 10D shows non-neoplastic qualities present in high-attention patches, as determined by patch mode, for 49 pathologist reviewed bags, according to some embodiments.

### DESCRIPTION OF EXAMPLE EMBODIMENTS

Multiple-instance learning (MIL) is a machine-learning technique in which a MIL machine-learning model trains on inputs of sets of image instances (e.g., referred to as a “bag” of patches of pixels) as opposed to the individual image instances themselves. For example, a bag of patches of pixels may be structured and pre-processed in a manner in which class labels are assigned to the bags of patches of pixels for the purposes of training the MIL model. For example, a histopathology image, such as a hemotoxin and eosin (H&E) slide, may include a tissue sample, which may be further sequenced and analyzed. In such an instance, one or more biomarkers or gene alterations may be determined as being associated with the whole tissue sample as opposed to, for example, any specific tissue cells comprising the tissue sample. Thus, the MIL model may be particular suitable for analyzing and classifying bags of patches of pixels in histopathology images, which may often include very large and high-resolution images. However, because the bags of patches of pixels are each taken from the same histopathology image, traditional machine-learning model techniques of tracking data as the model trains on batches of varying images may be unsuitable and diminish the overall prediction performance of the model. It may be useful to provide techniques to improve MIL models for predicting slide-level class labels for a singular whole-slide image.

Accordingly, the present embodiments are directed toward one or more computing devices, methods, and non-transitory computer-readable media that may generate inference-phase-specific batch normalization parameters for a machine-learning model trained to predict a slide-level class label describing one or more gene alterations or other biomarkers based on a singular whole-slide histopathology image. For example, a multiple-instance learning convolutional neural network (MILCNN) may include, for example, one or more convolutional layers, one or more pooling or max-pooling layers, one or more fully-connected layers, one or more batch normalization layers, and one or more rectified linear units (ReLUs). During a training phase of the MILCNN, the one or more batch normalization layers may utilize one or more sets of mini-batch normalization parameters (e.g., a mini-mean parameter and a mini-variance parameter) to normalize features in a feature map at each layer of the MILCNN.

Specifically, during the training phase of the MILCNN, each batch of data is normalized by subtracting the batch mean and dividing by the square root of the batch variance. However, during an inference phase of the MILCNN, at least one bag of patches of pixels of a singular whole-slide histopathology image may be inputted to the MILCNN and the MILCNN may predict a slide-level label (e.g., predict a slide-level label based on the entire bag of patches of pixels as opposed to the individual patches of pixels constituting the bag). Thus, during the inference phase, the MILCNN may generate and utilize inference-phase-specific batch normalization parameters, such that the one or more batch normalization layers may normalize features in a feature map at each feature layer of the MILCNN utilizing the inference-phase-specific batch normalization parameters as opposed to utilizing, for example, a running mean and variance calculated based on the one or more sets of mini-batch normalization parameters (e.g., a mini-mean parameter and a mini-variance parameter) learned during the training phase of the MILCNN.

By generating inference-phase-specific batch normalization statistics for the MILCNN, the trained MILCNN may better predict slide-level class labels describing one or more gene alterations or other biomarkers (e.g., an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, neurotrophic tyrosine receptor kinase (NTRK) genes 1/2/3, and so forth) based on a singular whole slide histopathology image. Particularly, in accordance with the presently-disclosed embodiments, by generating inference-phase-specific batch normalization parameters, the trained MILCNN may include batch normalization parameters that are appropriately “fitted” to the data, which includes bags of patches of pixels all corresponding to a singular whole-slide histopathology image (e.g., as opposed to being overly “fitted” to only the most recent images inputted to the MIL model as would otherwise be the case utilizing training-phase-determined running mean and running variance batch normalization parameters).

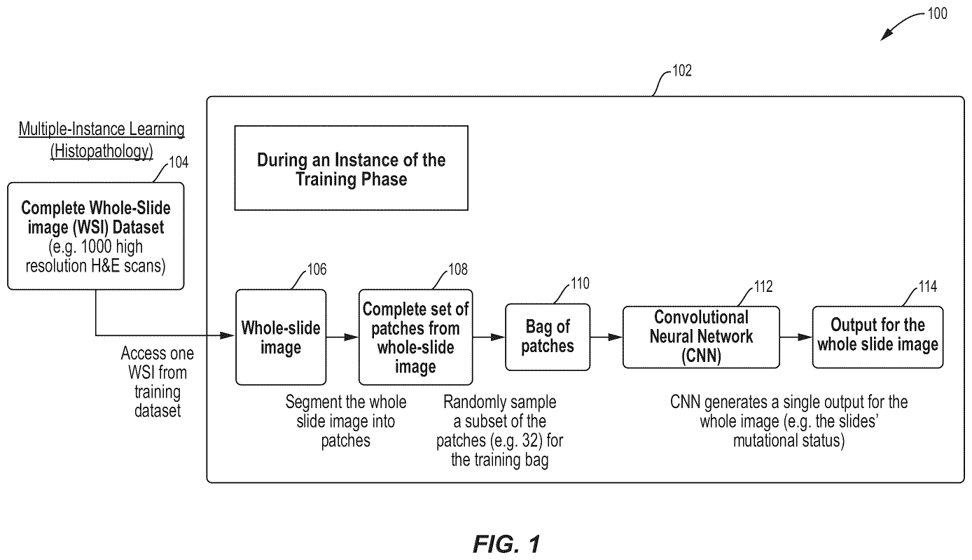

FIG. 1 illustrates a workflow diagram 100 of a training phase for training a machine-learning model to predict a slide-level class label describing one or more gene alterations or other biomarkers based on a singular whole-slide histopathology image, in accordance with the disclosed embodiments. In certain embodiments, the workflow diagram 100 may be performed by a MIL neural network pipeline 102. For example, in one embodiment, the MIL neural network pipeline 102 may be based on a residual neural network (ResNet) image-classification network or a deep ResNet image-classification network (e.g., e.g., ResNet-18, ResNet-34, ResNet-50, ResNet-101, ResNet-152) trained on a dataset based on natural (e.g., non-medical) images, such as the ImageNet dataset (e.g., a labeled high-resolution image database available publicly).

In certain embodiments, a data set of images 104 (e.g., histopathology images) may be accessed. In certain embodiments, the data set of images 104 may include, for example, any of various whole-slide images (WSIs), such as fluorescence in situ hybridization (FISH) images, an immunofluorescence (IF) images, hematoxylin and eosin (H&E) images, immunohistochemistry (IHC) images, imaging mass cytometry (IMC) images, and so forth. In one embodiment, the data set of images 104 may include a set of histopathology images (e.g., 1,000 or more histopathology images), which may each include very large and high-resolution images (e.g., 1.5K×2K pixels, 2K×4K pixels, 6K×8K pixels, 7.5K×10K pixels, 9K×12K pixels, 15K×20K pixels, 20K×24K pixels, 20K×30K pixels, 24K×30K pixels). The data set of images 104 are not limited to histopathology images, and may include any large and/or high-resolution images.

In certain embodiments, the MIL neural network pipeline 102 may be trained on a singular WSI 106 (per training instance or per training step) selected from the data set of images 104. Specifically, while previous techniques of training neural networks are based on batches of training images (e.g., 30-35 images per batch) each being independent of each other, in accordance with the presently-disclosed embodiments, the MIL neural network pipeline 102 may be trained on bags of patches of pixels all sampled from the same singular WSI 106. As further depicted, in certain embodiments, the MIL neural network pipeline 102 may further include segmenting the singular WSI 106 into a complete set of patches of pixels 108, which may each include different regions of pixels of the singular WSI 106 clustered into a respective patch.

In certain embodiments, the MIL neural network pipeline 102 may further include grouping the complete set of patches of pixels 108 into bags of patches of pixels 110. For example, in some embodiments, the MIL neural network pipeline 102 may include randomly sampling one or more subsets of the complete set of patches of pixels 108 patches (e.g., 30-35 patches of pixels) to be grouped or clustered into the bags of patches of pixels 110 for inputting into a MIL convolutional neural network (CNN) 112 for training the MILCNN model 112. In certain embodiments, MILCNN model 112 may include, for example, any multiple-learning neural network machine-learning model that may be trained to predict slide-level class labels based on the bags of patches of pixels 110, in which each bag of the bags of patches of pixels 110 includes that same training class label.

In certain embodiments, at least one bag of the bags of patches of pixels 110 may be inputted to the MILCNN model 112 to train the MILCNN model 112 to generate a prediction of a slide-level label 114 (e.g., generate a prediction a slide-level label based on the entire at least one bag of the bags of patches of pixels 110 as opposed to the individual subset of patches of pixels 108 patches constituting the bags of patches of pixels 110). In certain embodiments, the prediction of a slide-level label 114 may include, for example, a prediction of a slide-level class label describing one or more gene alterations or other biomarkers that may be included in the singular WSI 106. In certain embodiments, the workflow diagram 100 may be performed iteratively for each of the bags of patches of pixels 110 until the MILCNN model 112 is sufficiently trained (e.g., correctly predicting the prediction of a slide-level label 114 with a probability greater than 0.8 or greater than 0.9).

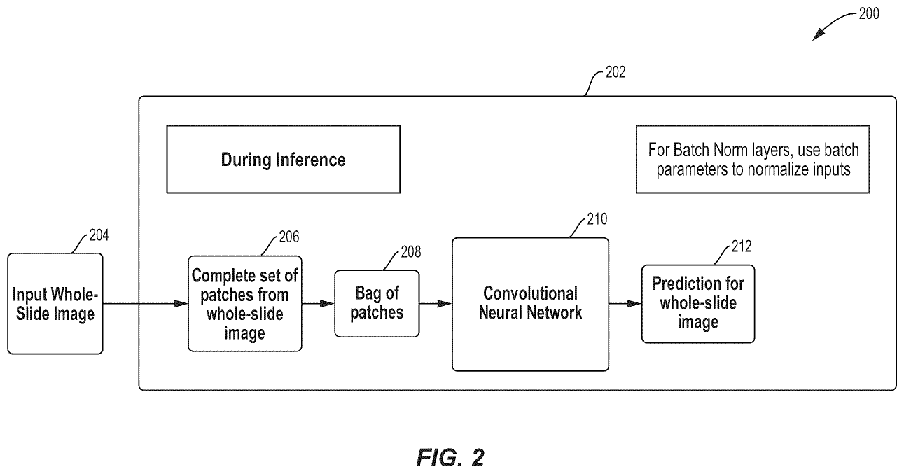

FIG. 2 illustrates a workflow diagram 200 of an inference phase for utilizing a machine-learning model trained to predict a slide-level class label describing one or more gene alterations or other biomarkers based on a singular whole-slide histopathology image, in accordance with the disclosed embodiments. It will be understood that the slide-level class label is not limited thereto and may be any label relevant to the image utilized to train the machine-learning model and to predict the slide-level class label. In certain embodiments, the workflow diagram 200 may be performed by a MIL neural network pipeline 202 (e.g., corresponding to the MIL neural network pipeline 102 having been trained as discussed above with respect to FIG. 1). In certain embodiments, an input WSI 204 may be accessed. In certain embodiments, as discussed above respect to FIG. 1, the input WSI 204 may include, for example, a fluorescence in situ hybridization (FISH) input image, an immunofluorescence (IF) input image, a hematoxylin and eosin (H&E) input image, immunohistochemistry (IHC) input image, an imaging mass cytometry (IMC) input image, and so forth.

In certain embodiments, the MIL neural network pipeline 202 may access a singular input WSI 204 (e.g., a real-world, clinical WSI). In accordance with the presently-disclosed embodiments, because the MIL neural network pipeline 102 was trained on the bags of patches of pixels 110 all sampled from the same singular WSI 106, previously techniques of batch normalization, in which each batch normalization layer in the network calculates its corresponding batch normalization parameters (e.g., mini-batch normalization parameters) during the training phase of the MILCNN model 112, for example, and then during the inference phase of the MILCNN model 210 (e.g., corresponding to the MIL neural network pipeline 102 having been trained as discussed above with respect to FIG. 1) batch normalization is performed based on a running mean and variance calculated based on the batch normalization parameters (e.g., mini-batch normalization parameters) determined during the training phase of the MILCNN model 112 may be unsuitable and diminish the overall predict performance of the MILCNN model 210, for example.

During the training phase of the MILCNN model 112, the running mean and variance of all of the bags of patches of pixels 110 may be calculated to be utilized during the inference phase of the MILCNN model 210 (e.g., corresponding to the MIL neural network pipeline 102 having been trained as discussed above with respect to FIG. 1). However, as previously noted, due to the nature of very large, high-resolution histopathology images (e.g., FISH images, IF images, H&E images, IHC images, IMC images, and so forth), which may involve training and analyzing bags of patches of pixels 110 all sampled from the same singular WSI 106, a calculated running mean and variance that converges to a population mean and variance may be unsuitable during the inference phase of the MILCNN model 210 since the MILCNN model 210 would be overly “fitted” to only the most recent bags of patches of pixels 110 patches inputted to the MILCNN model 112.

As further depicted, in certain embodiments, the MIL neural network pipeline 202 may further include segmenting the input WSI 204 into a complete set of patches of pixels 206, which may each include different regions of pixels of the input WSI 204 clustered into a respective patch. In certain embodiments, the MIL neural network pipeline 202 may further include grouping the complete set of patches of pixels 206 into bags of patches of pixels 208. For example, in some embodiments, the MIL neural network pipeline 202 may include randomly sampling one or more subsets of the complete set of patches of pixels 206 patches (e.g., 30-35 patches of pixels) to be grouped or clustered into the bags of patches of pixels 208 for inputting into the MILCNN model 210 (e.g., corresponding to the MIL neural network pipeline 102 having been trained as discussed above with respect to FIG. 1).

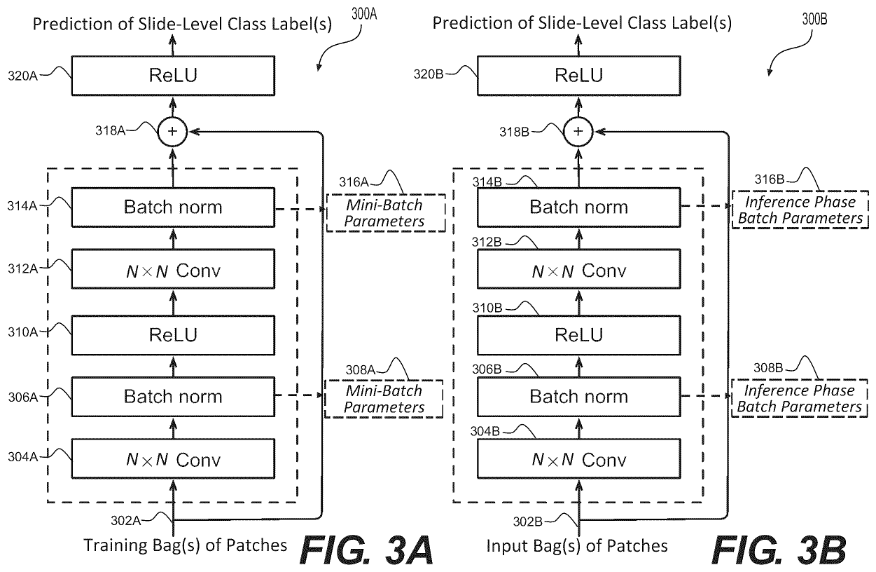

Accordingly, as will be further illustrated with respect to FIGS. 3A and 3B below, in accordance with the presently-disclosed embodiments, the MILCNN model 210 may generate and utilize inference-phase-specific batch normalization parameters, such that one or more batch normalization layers included as part of the MILCNN model 210 may normalize features in a feature map at each layer of the MILCNN model 210 utilizing the inference-phase-specific batch normalization parameters (e.g., as opposed to utilizing, for example, a running mean and variance calculated based on the set of mini-batch normalization parameters determined during the training phase of the MILCNN model 112).

In certain embodiments, at least one bag of the bags of patches of pixels 208 may be inputted to the MILCNN model 210 (e.g., corresponding to the MIL neural network pipeline 102 having been trained as discussed above with respect to FIG. 1) to generate a prediction of a single image level label 212 (e.g., a slide-level label) (e.g., generate a prediction a slide-level label based on the entire at least one bag of the bags of patches of pixels 208 as opposed to the individual subset of patches of pixels 206 patches constituting the bags of patches of pixels 208). In certain embodiments, the prediction of a slide-level label 114 may include, for example, a prediction of a slide-level class label describing one or more gene alterations or other biomarkers that may be included in the input WSI 204. For example, in some embodiments, the slide-level label 212 describing one or more gene alterations or other biomarkers may describe one or more gene alterations or other biomarkers including, for example, an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, neurotrophic tyrosine receptor kinase (NTRK) genes 1/2/3, and so forth.

FIGS. 3A and 3B illustrate a training phase and an inference phase of a multiple-instance learning (MIL) convolutional neural network (CNN) model 300A, 300B trained to predict a slide-level class label describing one or more gene alterations or other biomarkers based on a singular whole-slide histopathology image, in accordance with the disclosed embodiments. As depicted, in accordance with the presently disclosed embodiments, the MILCNN models 300A, 300B may include, for example, an ResNet image-classification network or a deep ResNet image-classification network (e.g., ResNet-18, ResNet-34, ResNet-50, ResNet-101, ResNet-152) trained and utilized as discussed above with respect to the MILCNN model 112 of FIG. 1 and the MILCNN model 210 of FIG. 2, respectively.

It should be appreciated that the MILCNN models 300A, 300B may represent only one example embodiment of a neural network architecture that may be trained and utilized to predict a slide-level class label describing one or more gene alterations or other biomarkers based on a singular whole-slide histopathology image in accordance with the presently disclosed embodiments. For example, in other embodiments, the MILCNN models 300A, 300B may include, for example, any number of convolutional layers, pooling layers or max-pooling layers, full-connected layers, batch normalization layers, or output layers (e.g., ReLUs, artificial neural network (ANN) classifiers, and so forth) that may be scaled-up or scaled-down based on the implementation and application.

In certain embodiments, during the training phase, the MILCNN model 300A may receive an input 302A including a training bag of patches of pixels. In certain embodiments, the input 302A including the training bag of patches of pixels may be inputted in a first N×N convolutional layer 304A (e.g., a 3×3 convolutional layer, a 7×7 convolutional layer, 10×10 convolutional layer). For example, in some embodiments, the first N×N convolutional layer 304A may include, for example, one or more convolutional filters or kernels that may be utilized to extract and generate a feature map based on the input 302A including a training bag of patches of pixels. Although not illustrated, in certain embodiments, one or more weighing layers may be included as part of the first N×N convolutional layer 304A (e.g., included as part of the one or more full-connected layers) that may be utilized to generate and iteratively update one or more gamma parameters (i.e., scaling parameters) and beta parameters (i.e., offset parameters) that may be associated with weighting and biasing the MILCNN model 300A.

In certain embodiments, the feature map outputted by the first N×N convolutional layer 304A may be then inputted to a first batch normalization layer 306A. For example, the first batch normalization layer 306A may normalize the feature map utilizing a first set of mini-batch parameters 308A. In certain embodiments, the first set of mini-batch parameters 308A (e.g., mini mean value and mini variance value) may include, for example, batch normalization statistics that correspond to the current training bag of patches of pixels inputted to the first N×N convolutional layer 304A and the first feature layer of the MILCNN model 300A. In certain embodiments, the first batch normalization layer 306A may then output the normalized feature map to a first output layer 310A (e.g., first ReLU layer or first activation function layer), which may be utilized to generate a first probability or probability distribution based on the normalized feature map.

In certain embodiments, the first N×N convolutional layer 304A, the first batch normalization layer 306A, and the first output layer 310A (e.g., first ReLU layer or first activation function layer) may collectively represent a first feature layer of the MILCNN model 300A tasked with learning one or more first specific image features of the input 302A including a training bag of patches of pixels (e.g., first feature layer of the MILCNN model 300A may learn and predict edges, the second feature layer of the MILCNN model 300A may learn and predict contours, the third feature layer of the MILCNN model 300A may learn and predict surfaces, and so on and so forth). In certain embodiments, the first probability or probability distribution outputted by the first output layer 310A (e.g., first ReLU layer or first activation function layer) may be inputted to a second N×N convolutional layer 312A (e.g., a 3×3 convolutional layer, a 7×7 convolutional layer, 10×10 convolutional layer).

In certain embodiments, the second N×N convolutional layer 312A may then generate a second feature map based on the input 302A including a training bag of patches of pixels and the first probability or probability distribution outputted by the first output layer 310A (e.g., first ReLU layer or first activation function layer). In certain embodiments, the second feature map outputted by the second N×N convolutional layer 312A may be then inputted to a second batch normalization layer 314A. For example, the second batch normalization layer 314A may normalize the second feature map utilizing a second set of mini-batch parameters 316A. In certain embodiments, the second set of mini-batch parameters 316A (e.g., mini mean value and mini variance value) may include, for example, batch normalization statistics that correspond to the current training bag of patches of pixels inputted to the first N×N convolutional layer 304A and the second feature layer of the MILCNN model 300A.

In certain embodiments, the second batch normalization layer 314A may then output the second normalized feature map to a second output layer 318A (e.g., second ReLU layer or second activation function layer), which may be utilized to generate a second probability or probability distribution based on the second normalized feature map. The second probability or probability distribution outputted by the second output layer 318A (e.g., second ReLU layer or second activation function layer) and the input 302A including a training bag of patches of pixels may be then summed (e.g., via a summer 320A) and outputted to a third output layer 322A (e.g., third ReLU layer or third activation function layer). In certain embodiments, the third output layer 322A (e.g., third ReLU layer or third activation function layer) may then generate a final prediction of a slide-level class label describing one or more gene alterations or other biomarkers that may be included in a WSI from which the input 302A including a training bag of patches of pixels was sampled.

In certain embodiments, during the inference phase, the MILCNN model 300B may receive an input 302B including an input bag of patches of pixels. In certain embodiments, the input 302B including the input bag of patches of pixels may be inputted in a first N×N convolutional layer 304B (e.g., a 3×3 convolutional layer, a 7×7 convolutional layer, 10×10 convolutional layer). For example, in some embodiments, the first N×N convolutional layer 304B may include, for example, one or more convolutional filters or kernels that may be utilized to extract and generate a feature map based on the input 302B including an input bag of patches of pixels. As previously noted with respect to FIG. 3A, although not illustrated, in certain embodiments, one or more weighing layers may be included as part of the first N×N convolutional layer 304B (e.g., included as part of the one or more full-connected layers) that may be utilized to generate and iteratively update one or more gamma parameters (i.e., scaling parameters) and beta parameters (i.e., offset parameters) that may be associated with weighting and biasing the MILCNN model 300B.

In certain embodiments, the feature map outputted by the first N×N convolutional layer 304B may be then inputted to a first batch normalization layer 306B. For example, in accordance with the presently disclosed embodiments, the first batch normalization layer 306B may then generate a first set of inference-phase-specific batch normalization parameters 308B (e.g., inference-phase-specific mean value and an inference-phase-specific variance value). For example, the set of inference-phase-specific batch normalization parameters 308B may be then utilized to normalize the feature map outputted by the first N×N convolutional layer 304B. In certain embodiments, the first set of inference-phase-specific batch normalization parameters 308B (e.g., inference-phase-specific mean value and an inference-phase-specific variance value) may include, for example, batch normalization statistics that correspond to the current input bag of patches of pixels inputted to the first N×N convolutional layer 304B and the first feature layer of the MILCNN model 300B. In certain embodiments, the first batch normalization layer 306B may then output the normalized feature map to a first output layer 310B (e.g., first ReLU layer or first activation function layer), which may be utilized to generate a first probability or probability distribution based on the normalized feature map outputted by the first batch normalization layer 306B.

In certain embodiments, the first N×N convolutional layer 304B, the first batch normalization layer 306B, and the first output layer 310B (e.g., first ReLU layer or first activation function layer) may collectively represent a first feature layer of the MILCNN model 300B tasked with learning one or more first specific image features of the input 302B including an input bag of patches of pixels (e.g., first feature layer of the MILCNN model 300B may predict edges, the second feature layer of the MILCNN model 300B may predict contours, the third feature layer of the MILCNN model 300B may predict surfaces, and so on and so forth). In certain embodiments, the first probability or probability distribution outputted by the first output layer 310B (e.g., first ReLU layer or first activation function layer) may be inputted to a second N×N convolutional layer 312B (e.g., a 3×3 convolutional layer, a 7×7 convolutional layer, 10×10 convolutional layer).

In certain embodiments, the second N×N convolutional layer 312B may then generate a second feature map based on the input 302B including an input bag of patches of pixels and the first probability or probability distribution outputted by the first output layer 310B (e.g., first ReLU layer or first activation function layer). In certain embodiments, the second feature map outputted by the second N×N convolutional layer 312B may be then inputted to a second batch normalization layer 314B. For example, in accordance with the presently disclosed embodiments, the second batch normalization layer 314B may then generate a second set of inference-phase-specific batch normalization parameters 314B (e.g., inference-phase-specific mean value and an inference-phase-specific variance value). For example, the set of inference-phase-specific batch normalization parameters 314B may be then utilized to normalize the feature map outputted by the second N×N convolutional layer 312B.

In certain embodiments, the second batch normalization layer 314B may then output the second normalized feature map to a second output layer 318B (e.g., second ReLU layer or second activation function layer), which may be utilized to generate a second probability or probability distribution based on the second normalized feature map. The second probability or probability distribution outputted by the second output layer 318B (e.g., second ReLU layer or second activation function layer) and the input 302B including an input bag of patches of pixels may be then summed (e.g., via a summer 320B) and outputted to a third output layer 322B (e.g., third ReLU layer or third activation function layer). In certain embodiments, the third output layer 322B (e.g., third ReLU layer or third activation function layer) may then generate a final prediction of a slide-level class label describing one or more gene alterations or other biomarkers that may be included in a WSI from which the input 302B including an input bag of patches of pixels was sampled.

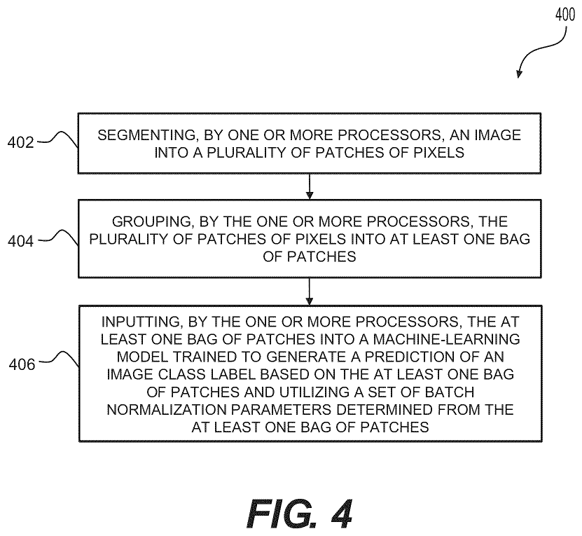

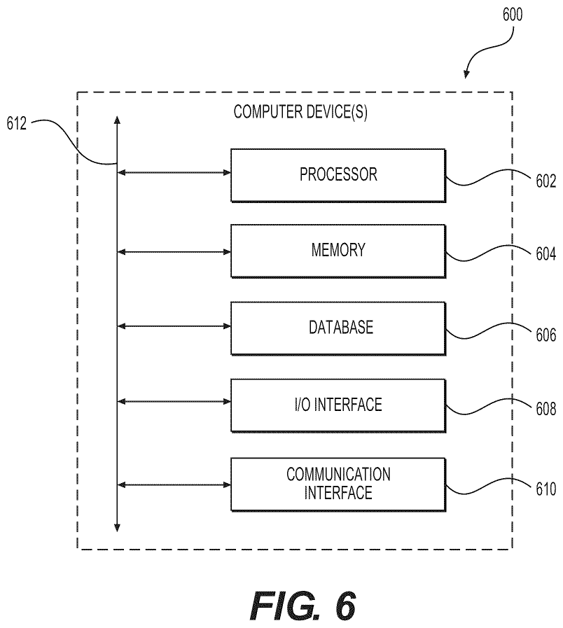

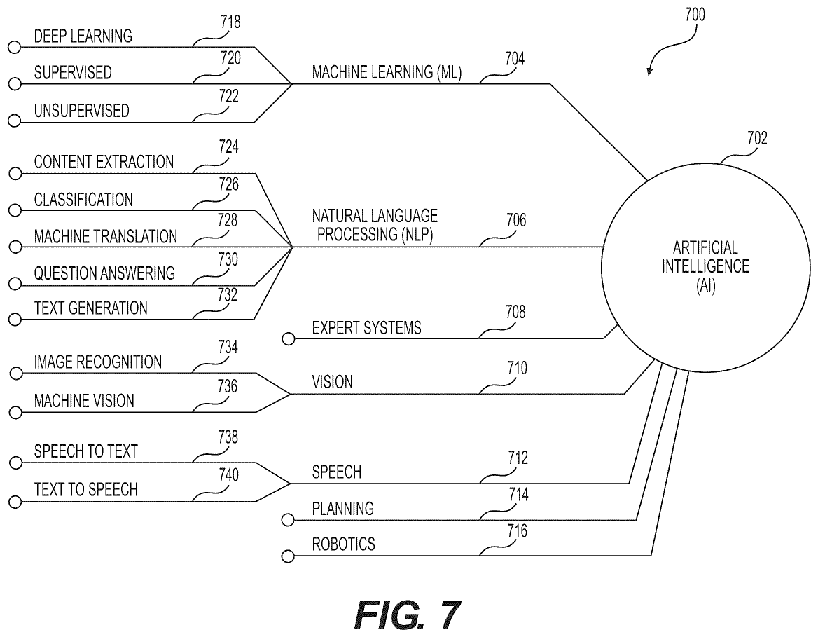

FIG. 4 illustrates a flow diagram of a method 400 for utilizing a machine-learning model train to predict a slide-level class label describing one or more gene alterations or other biomarkers based on a singular whole-slide histopathology image, in accordance with the disclosed embodiments. The method 400 may be performed utilizing one or more processing devices (e.g., computing device(s) and artificial intelligence architecture to be discussed below with respect to FIGS. 6 and 7) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various omics data and making one or more decisions based thereon), software (e.g., instructions running/executing on one or more processors), firmware (e.g., microcode), or some combination thereof.

The method 400 may begin at block 402 with one or more processing devices segmenting an image into a plurality of patches. The method 400 may then continue at block 404 with one or more processing devices grouping the plurality of patches into at least one bag of patches. The method 400 may then conclude at block 406 with one or more processing devices inputting the at least one bag of patches into a machine-learning model trained to generate a prediction of an image class label based on the at least one bag of patches and utilizing a set of batch normalization parameters determined from the at least one bag of patches.

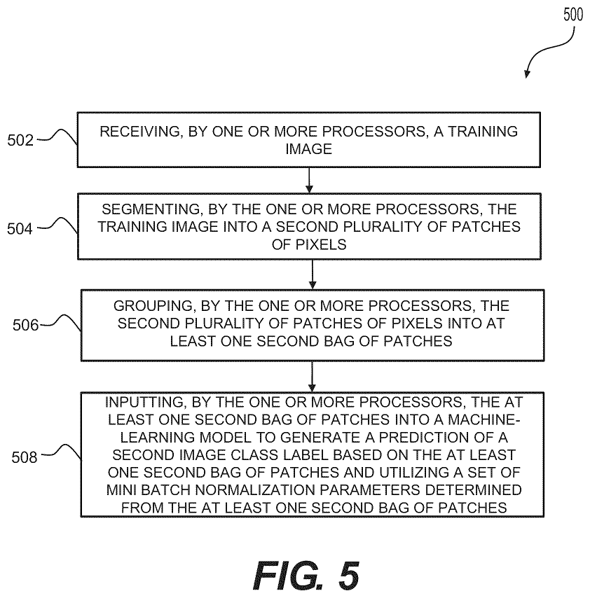

FIG. 5 illustrates a flow diagram of a method 500 for training a machine-learning model to predict an image label (e.g., a slide-level class label) describing one or more gene alterations or other biomarkers based on a singular image (e.g., a whole-slide histopathology image), in accordance with the disclosed embodiments. The method 500 may be performed utilizing one or more processing devices (e.g., computing device(s) and artificial intelligence architecture to be discussed below with respect to FIGS. 6 and 7) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various omics data and making one or more decisions based thereon), software (e.g., instructions running/executing on one or more processors), firmware (e.g., microcode), or some combination thereof.

The method 500 may begin at block 502 with one or more processing devices receiving a training image. The method 500 may then continue at block 504 with one or more processing devices segmenting the training image into a second plurality of patches of pixels. The method 500 may then continue at block 506 with one or more processing devices grouping the plurality of patches of pixels into at least one second bag of patches. The method 500 may then conclude at block 508 with one or more processing devices inputting the at least one second bag of patches into a machine-learning model to generate a prediction of an image class label based on the at least one second bag of patches and utilizing a set of mini-batch normalization parameters determined from the at least one second bag of patches.

Accordingly, as generally set forth by the method 400 of FIG. 4 and the method 500 of FIG. 5, the present embodiments are directed toward one or more computing devices, methods, and non-transitory computer-readable media that may generate inference-phase-specific batch normalization parameters for a machine-learning model trained to predict a label (e.g., a slide-level class) describing a portion of the image (e.g., one or more gene alterations or other biomarkers) based on a singular image (e.g., a singular whole-slide histopathology image). For example, a multiple-instance learning convolutional neural network (MILCNN) may include, for example, one or more convolutional layers, one or more pooling or max-pooling layers, one or more fully-connected layers, one or more batch normalization layers, and one or more rectified linear units (ReLUs). During a training phase of the MILCNN, the one or more batch normalization layers may utilize one or more sets of mini-batch normalization parameters (e.g., a mini-mean parameter and a mini-variance parameter) to normalize features in a feature map at each layer of the MILCNN.

Specifically, during the training phase of the MILCNN, each batch of data is normalized by subtracting the batch mean and dividing by the square root of the batch variance. However, during an inference phase of the MILCNN, at least one bag of patches of pixels of a singular image (e.g., a singular whole-slide histopathology image) may be inputted to the MILCNN and the MILCNN may predict an image-level label (e.g., predict a slide-level label based on the entire bag of patches of pixels as opposed to the individual patches of pixels constituting the bag). Thus, during the inference phase, the MILCNN may generate and utilize inference-phase-specific batch normalization parameters, such that the one or more batch normalization layers may normalize features in a feature map at each feature layer of the MILCNN utilizing the inference-phase-specific batch normalization parameters as opposed to utilizing, for example, a running mean and variance calculated based on the one or more sets of mini-batch normalization parameters (e.g., a mini-mean parameter and a mini-variance parameter) learned during the training phase of the MILCNN.

By generating inference-phase-specific batch normalization statistics for the MILCNN, the trained MILCNN may better predict slide-level class labels describing one or more gene alterations or other biomarkers (e.g., an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration) based on a singular whole slide histopathology image. Particularly, in accordance with the presently-disclosed embodiments, by generating inference-phase-specific batch normalization parameters, the trained MILCNN may include batch normalization parameters that are appropriately “fitted” to the data, which includes bags of patches of pixels all corresponding to a singular whole-slide histopathology image (e.g., as opposed to being overly “fitted” to only the most recent images inputted to the MIL model as would otherwise be the case utilizing training-phase-determined running mean and running variance batch normalization parameters).

FIG. 6 illustrates an example of one or more computing device(s) 600 that may be utilized to generate inference-phase-specific batch normalization parameters for a machine-learning model trained to predict a slide-level class label describing one or more gene alterations or other biomarkers based on a singular whole-slide histopathology image, in accordance with the disclosed embodiments. In certain embodiments, the one or more computing device(s) 600 may perform one or more steps of one or more methods described or illustrated herein. In certain embodiments, the one or more computing device(s) 600 provide functionality described or illustrated herein. In certain embodiments, software running on the one or more computing device(s) 600 performs one or more steps of one or more methods described or illustrated herein, or provides functionality described or illustrated herein. Certain embodiments include one or more portions of the one or more computing device(s) 600.

This disclosure contemplates any suitable number of computing systems 600. This disclosure contemplates one or more computing device(s) 600 taking any suitable physical form. As example and not by way of limitation, one or more computing device(s) 600 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the one or more computing device(s) 600 may be unitary or distributed; span multiple locations; span multiple machines; span multiple data centers; or reside in a cloud, which may include one or more cloud components in one or more networks.

Where appropriate, the one or more computing device(s) 600 may perform without substantial spatial or temporal limitation one or more steps of one or more methods described or illustrated herein. As an example, and not by way of limitation, the one or more computing device(s) 600 may perform, in real-time or in batch mode, one or more steps of one or more methods described or illustrated herein. The one or more computing device(s) 600 may perform, at different times or at different locations, one or more steps of one or more methods described or illustrated herein, where appropriate.

In certain embodiments, the one or more computing device(s) 600 includes a processor 602, memory 604, database 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement. In certain embodiments, processor 602 includes hardware for executing instructions, such as those making up a computer program. As an example, and not by way of limitation, to execute instructions, processor 602 may retrieve (or fetch) the instructions from an internal register, an internal cache, memory 604, or database 606; decode and execute them; and then write one or more results to an internal register, an internal cache, memory 604, or database 606. In certain embodiments, processor 602 may include one or more internal caches for data, instructions, or addresses. This disclosure contemplates processor 602 including any suitable number of any suitable internal caches, where appropriate. As an example, and not by way of limitation, processor 602 may include one or more instruction caches, one or more data caches, and one or more translation lookaside buffers (TLBs). Instructions in the instruction caches may be copies of instructions in memory 604 or database 606, and the instruction caches may speed up retrieval of those instructions by processor 602.

Data in the data caches may be copies of data in memory 604 or database 606 for instructions executing at processor 602 to operate on; the results of previous instructions executed at processor 602 for access by subsequent instructions executing at processor 602 or for writing to memory 604 or database 606; or other suitable data. The data caches may speed up read or write operations by processor 602. The TLBs may speed up virtual-address translation for processor 602. In certain embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In certain embodiments, memory 604 includes main memory for storing instructions for processor 602 to execute or data for processor 602 to operate on. As an example, and not by way of limitation, the one or more computing device(s) 600 may load instructions from database 606 or another source (such as, for example, another one or more computing device(s) 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604.

In certain embodiments, processor 602 executes only instructions in one or more internal registers, internal caches, or memory 604 (as opposed to database 606 or elsewhere) and operates only on data in one or more internal registers, internal caches, or memory 604 (as opposed to database 606 or elsewhere). One or more memory buses (which may each include an address bus and a data bus) may couple processor 602 to memory 604. Bus 612 may include one or more memory buses, as described below. In certain embodiments, one or more memory management units (MMUs) reside between processor 602 and memory 604 and facilitate accesses to memory 604 requested by processor 602. In certain embodiments, memory 604 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memory devices 604, where appropriate. Although this disclosure describes and illustrates particular memory, this disclosure contemplates any suitable memory.

In certain embodiments, database 606 includes mass storage for data or instructions. As an example, and not by way of limitation, database 606 may include a hard disk drive (HDD), a floppy disk drive, flash memory, an optical disc, a magneto-optical disc, magnetic tape, or a Universal Serial Bus (USB) drive, or a combination of two or more of these. Database 606 may include removable or non-removable (or fixed) media, where appropriate. Database 606 may be internal or external to the one or more computing device(s) 600, where appropriate. In certain embodiments, database 606 is non-volatile, solid-state memory. In certain embodiments, database 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), flash memory, or a combination of two or more of these. This disclosure contemplates mass database 606 taking any suitable physical form. Database 606 may include one or more storage control units facilitating communication between processor 602 and database 606, where appropriate. Where appropriate, database 606 may include one or more databases 606. Although this disclosure describes and illustrates particular storage, this disclosure contemplates any suitable storage.

In certain embodiments, I/O interface 608 includes hardware, software, or both, providing one or more interfaces for communication between the one or more computing device(s) 600 and one or more I/O devices. The one or more computing device(s) 600 may include one or more of these I/O devices, where appropriate. One or more of these I/O devices may enable communication between a person and the one or more computing device(s) 600. As an example, and not by way of limitation, an I/O device may include a keyboard, keypad, microphone, monitor, mouse, printer, scanner, speaker, still camera, stylus, tablet, touch screen, trackball, video camera, another suitable I/O device, or a combination of two or more of these. An I/O device may include one or more sensors. This disclosure contemplates any suitable I/O devices and any suitable I/O interfaces 608 for them. Where appropriate, I/O interface 608 may include one or more device or software drivers enabling processor 602 to drive one or more of these I/O devices. I/O interface 608 may include one or more I/O interfaces 608, where appropriate. Although this disclosure describes and illustrates a particular I/O interface, this disclosure contemplates any suitable I/O interface.

In certain embodiments, communication interface 610 includes hardware, software, or both providing one or more interfaces for communication (such as, for example, packet-based communication) between the one or more computing device(s) 600 and one or more other computing device(s) 600 or one or more networks. As an example, and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it.

As an example, and not by way of limitation, the one or more computing device(s) 600 may communicate with an ad hoc network, a personal area network (PAN), a local area network (LAN), a wide area network (WAN), a metropolitan area network (MAN), one or more portions of the Internet, or a combination of two or more of these. One or more portions of one or more of these networks may be wired or wireless. As an example, the one or more computing device(s) 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), other suitable wireless network, or a combination of two or more of these. The one or more computing device(s) 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In certain embodiments, bus 612 includes hardware, software, or both coupling components of the one or more computing device(s) 600 to each other. As an example, and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, another suitable bus, or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

FIG. 7 illustrates a diagram 700 of an example artificial intelligence (AI) architecture 702 (which may be included as part of the one or more computing device(s) 600 as discussed above with respect to FIG. 6) that may be utilized to generate inference-phase-specific batch normalization parameters for a machine-learning model trained to predict a slide-level class label describing one or more gene alterations or other biomarkers based on a singular whole-slide histopathology image, in accordance with the disclosed embodiments. In certain embodiments, the AI architecture 702 may be implemented utilizing, for example, one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), and/or other processing device(s) that may be suitable for processing various molecular data and making one or more decisions based thereon), software (e.g., instructions running/executing on one or more processing devices), firmware (e.g., microcode), or some combination thereof.

In certain embodiments, as depicted by FIG. 7, the AI architecture 702 may include machine learning (ML) algorithms and functions 704, natural language processing (NLP) algorithms and functions 706, expert systems 708, computer-based vision algorithms and functions 710, speech recognition algorithms and functions 712, planning algorithms and functions 714, and robotics algorithms and functions 716. In certain embodiments, the ML algorithms and functions 704 may include any statistics-based algorithms that may be suitable for finding patterns across large amounts of data (e.g., “Big Data” such as genomics data, proteomics data, metabolomics data, metagenomics data, transcriptomics data, or other omics data). For example, in certain embodiments, the ML algorithms and functions 704 may include deep learning algorithms 718, supervised learning algorithms 720, and unsupervised learning algorithms 722.

In certain embodiments, the deep learning algorithms 718 may include any artificial neural networks (ANNs) that may be utilized to learn deep levels of representations and abstractions from large amounts of data. For example, the deep learning algorithms 718 may include ANNs, such as a perceptron, a multilayer perceptron (MLP), an autoencoder (AE), a convolution neural network (CNN), a recurrent neural network (RNN), long short term memory (LSTM), a grated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), a spiking neural network (SNN), deep reinforcement learning, and so forth.

In certain embodiments, the supervised learning algorithms 720 may include any algorithms that may be utilized to apply, for example, what has been learned in the past to new data using labeled examples for predicting future events. For example, starting from the analysis of a known data set, the supervised learning algorithms 720 may produce an inferred function to make predictions about the output values. The supervised learning algorithms 600 may also compare its output with the correct and intended output and find errors in order to modify the supervised learning algorithms 720 accordingly. On the other hand, the unsupervised learning algorithms 722 may include any algorithms that may applied, for example, when the data used to train the unsupervised learning algorithms 722 are neither classified nor labeled. For example, the unsupervised learning algorithms 722 may study and analyze how systems may infer a function to describe a hidden structure from unlabeled data.

In certain embodiments, the NLP algorithms and functions 706 may include any algorithms or functions that may be suitable for automatically manipulating natural language, such as speech and/or text. For example, in some embodiments, the NLP algorithms and functions 706 may include content extraction algorithms or functions 724, classification algorithms or functions 726, machine translation algorithms or functions 728, question answering (QA) algorithms or functions 730, and text generation algorithms or functions 732. In certain embodiments, the content extraction algorithms or functions 724 may include a means for extracting text or images from electronic documents (e.g., webpages, text editor documents, and so forth) to be utilized, for example, in other applications.

In certain embodiments, the classification algorithms or functions 726 may include any algorithms that may utilize a supervised learning model (e.g., logistic regression, naïve Bayes, stochastic gradient descent (SGD), k-nearest neighbors, decision trees, random forests, support vector machine (SVM), and so forth) to learn from the data input to the supervised learning model and to make new observations or classifications based thereon. The machine translation algorithms or functions 728 may include any algorithms or functions that may be suitable for automatically converting source text in one language, for example, into text in another language. The QA algorithms or functions 730 may include any algorithms or functions that may be suitable for automatically answering questions posed by humans in, for example, a natural language, such as that performed by voice-controlled personal assistant devices. The text generation algorithms or functions 732 may include any algorithms or functions that may be suitable for automatically generating natural language texts.

In certain embodiments, the expert systems 708 may include any algorithms or functions that may be suitable for simulating the judgment and behavior of a human or an organization that has expert knowledge and experience in a particular field (e.g., stock trading, medicine, sports statistics, and so forth). The computer-based vision algorithms and functions 710 may include any algorithms or functions that may be suitable for automatically extracting information from images (e.g., photo images, video images). For example, the computer-based vision algorithms and functions 710 may include image recognition algorithms 734 and machine vision algorithms 736. The image recognition algorithms 734 may include any algorithms that may be suitable for automatically identifying and/or classifying objects, places, people, and so forth that may be included in, for example, one or more image frames or other displayed data. The machine vision algorithms 736 may include any algorithms that may be suitable for allowing computers to “see”, or, for example, to rely on image sensors cameras with specialized optics to acquire images for processing, analyzing, and/or measuring various data characteristics for decision making purposes.

In certain embodiments, the speech recognition algorithms and functions 712 may include any algorithms or functions that may be suitable for recognizing and translating spoken language into text, such as through automatic speech recognition (ASR), computer speech recognition, speech-to-text (STT) 738, or text-to-speech (TTS) 740 in order for the computing to communicate via speech with one or more users, for example. In certain embodiments, the planning algorithms and functions 714 may include any algorithms or functions that may be suitable for generating a sequence of actions, in which each action may include its own set of preconditions to be satisfied before performing the action. Examples of AI planning may include classical planning, reduction to other problems, temporal planning, probabilistic planning, preference-based planning, conditional planning, and so forth. Lastly, the robotics algorithms and functions 716 may include any algorithms, functions, or systems that may enable one or more devices to replicate human behavior through, for example, motions, gestures, performance tasks, decision-making, emotions, and so forth.

The methods described herein may further be used to characterize a cancer in a subject, for example as having a positive biomarker status. For example, the method may be used to characterize the cancer as positive for a mutation or alteration in a genetic biomarker. The genetic biomarker may be, for example, an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, or one or more neurotrophic tyrosine receptor kinase (NTRK) genes 1/2/3.

In some instances, there is a method of treating a subject with cancer, comprising characterizing the cancer of the subject being positive for the genetic alteration according to the method described herein, and administering to the subject an effective therapy. The effective therapy may be, for example, a poly (ADP-ribose) polymerase inhibitor (PARPi), a platinum compound, a kinase inhibitor, chemotherapy, radiation therapy, a targeted therapy (e.g., immunotherapy), surgery, or any combination thereof.

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The term “automatically” and its derivatives means “without human intervention,” unless expressly indicated otherwise or indicated otherwise by context.

The embodiments disclosed herein are only examples, and the scope of this disclosure is not limited to them. Embodiments according to this disclosure are in particular disclosed in the attached claims directed to a method, a storage medium, a system and a computer program product, wherein any feature mentioned in one claim category, e.g. method, may be claimed in another claim category, e.g. system, as well. The dependencies or references back in the attached claims are chosen for formal reasons only. However, any subject matter resulting from a deliberate reference back to any previous claims (in particular multiple dependencies) may be claimed as well, so that any combination of claims and the features thereof are disclosed and may be claimed regardless of the dependencies chosen in the attached claims. The subject-matter which may be claimed comprises not only the combinations of features as set out in the attached claims but also any other combination of features in the claims, wherein each feature mentioned in the claims may be combined with any other feature or combination of other features in the claims. Furthermore, any of the embodiments and features described or depicted herein may be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages.

### EXEMPLARY EMBODIMENTS

The following embodiments are exemplary and are not intended to limit the scope of the claimed invention. Among the provided embodiments are:

Embodiment 1. A method, comprising:
segmenting, by one or more processors, an image into a plurality of patches;
grouping, by the one or more processors, the plurality of patches into at least one bag of patches;
inputting, by the one or more processors, the at least one bag of patches into a machine-learning model trained to generate a prediction of an image class label based on the at least one bag of patches, the machine-learning model including:
   a first layer trained to generate one or more feature maps based on the at least one bag of patches;
   a second layer trained to normalize the one or more feature maps utilizing a set of batch normalization parameters determined from the at least one bag of patches to generate one or more normalized feature maps; and
   a third layer trained to generate the prediction of the image class label based at least in part on the one or more normalized feature maps; and outputting, by the one or more processors, the prediction of the image class label.

Embodiment 2. The method of embodiment 1, wherein the image comprises only one whole-slide image (WSI).

Embodiment 3. The method of any one of embodiments 1 or 2, further comprising receiving, by the one or more processors, the image, wherein the image comprises an image of a tissue sample.

Embodiment 4. The method of any one of embodiments 1-3, wherein each patch of the plurality of patches comprises a plurality of pixels corresponding to one or more regions of the image.

Embodiment 5. The method of any one of embodiments 1-4, wherein the image comprises a histological stain image, a fluorescence in situ hybridization (FISH) image, an immunofluorescence (IF) image, or a hematoxylin and eosin (H&E) image.

Embodiment 6. The method of any one of embodiments 1-5, wherein:
the first layer comprises one or more convolutional layers;
the second layer comprises one or more batch normalization layers; and
the third layer comprises an output layer.

Embodiment 7. The method of any one of embodiments 1-6, wherein the machine-learning model further comprises a pooling layer and a fully connected layer.

Embodiment 8. The method of any one of embodiments 1-7, wherein the machine-learning model comprises one or more convolutional neural networks (CNNs).

Embodiment 9. The method of any one of embodiments 1-8, wherein the machine-learning model comprises a multiple-instance learning (MIL) machine-learning model.

Embodiment 10. The method of any one of embodiments 1-9, wherein the machine-learning model comprises a multiple-instance learning convolutional neural network (MILCNN) machine-learning model.

Embodiment 11. The method of any one of embodiments 1-10, wherein the set of batch normalization parameters comprises a mean and a variance determined from the at least one bag of patches.

Embodiment 12. The method of any one of embodiments 1-11, wherein the set of batch normalization parameters corresponds to only the at least one second bag of patches.

Embodiment 13. The method of any one of embodiments 1-12, wherein the machine-learning model was trained by:
receiving, by the one or more processors, a training image;
segmenting, by the one or more processors, the training image into a second plurality of patches;
grouping, by the one or more processors, the second plurality of patches into at least one second bag of patches; and
inputting, by the one or more processors, the at least one second bag of patches into the machine-learning model to generate a prediction of a second image class label based on the at least one second bag of patches;
wherein:
   the first layer is trained to generate one or more feature maps based on the at least one second bag of patches;
   the second layer is trained to normalize the one or more second feature maps utilizing a set of mini-batch normalization parameters determined from the at least one second bag of patches to generate one or more second normalized feature maps; and
   the third layer is trained to generate the prediction of the second image class label for the training image based at least in part on the one or more second normalized feature maps.

Embodiment 14. The method of any one of embodiments 1-13, wherein each patch of the second plurality of patches comprises a plurality of pixels corresponding to one or more regions of the training image.

Embodiment 15. The method of any one of embodiments 1-14, wherein:
the first layer comprises one or more convolutional layers;
the second layer comprises one or more batch normalization layers; and
the third layer comprises an output layer.

Embodiment 16. The method of any one of embodiments 1-15, wherein the one or more batch normalization layers are trained to compute at least one of a running mean, a running variance, a gamma parameter, and a beta parameter of each of a plurality of sets of mini-batch normalization parameters during a training phase of the machine-learning model.

Embodiment 17. The method of any one of embodiments 1-16, wherein, in response to the machine-learning model being trained, one or more of the gamma parameter and the beta parameter are fixed.

Embodiment 18. The method of any one of embodiments 1-17, wherein in response to the machine-learning model being trained, one or more of a mean and a variance determined for the at least one second bag of patches is configured to be determined for each additional bag of patches from the at least one second bag of patches.

Embodiment 19. The method of any one of embodiments 1-18, wherein the set of mini-batch normalization parameters comprises a mini-batch mean and a mini-batch variance.

Embodiment 20. The method of any one of embodiments 1-19, wherein segmenting the training image into at least one second bag of patches comprises randomly sampling one or more patches of pixels of the at least one second bag of patches.

Embodiment 21. The method of any one of embodiments 1-20, wherein the image class label comprises an indication of a genetic biomarker of a tissue sample captured in the image.

Embodiment 22. The method of any one of embodiments 1-21, wherein the genetic biomarker of the tissue sample comprises an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, or one or more neurotrophic tyrosine receptor kinase (NTRK) genes 1/2/3.

Embodiment 23. The method of any one of embodiments 1-22, further comprising generating a report based on the prediction of the image class label indicating the genetic biomarker of the tissue sample.

Embodiment 24. The method of any one of embodiments 1-23, further comprising causing one or more electronic devices to display the report.

Embodiment 25. The method of any one of embodiments 1-24, wherein causing the one or more electronic devices to display the report comprises causing a human machine interface (HMI) associated with a pathologist to display the report.

Embodiment 26. A system including one or more computing devices, comprising:
one or more non-transitory computer-readable storage media including instructions; and
one or more processors coupled to the one or more storage media, the one or more processors configured to execute the instructions to:
   segment an image into a plurality of patches;
   group the plurality of patches into at least one bag of patches; and
   input the at least one bag of patches into a machine-learning model trained to generate a prediction of an image class label based on the at least one bag of patches, the machine-learning model including:
      a first layer trained to generate one or more feature maps based on the at least one bag of patches;
      a second layer trained to normalize the one or more feature maps utilizing a set of batch normalization parameters determined from the at least one bag of patches to generate one or more normalized feature maps; and
      a third layer trained to generate the prediction of the image class label based at least in part on the one or more normalized feature maps; and
   output the prediction of the image class label.

Embodiment 27. The system of embodiment 26, wherein the image comprises only one whole-slide image (WSI).

Embodiment 28. The system of embodiment 26 or 27, wherein the instructions further comprise instructions to receive the image, wherein the image comprises an image of a tissue sample.

Embodiment 29. The system of any one of embodiments 26-28, wherein each patch of the plurality of patches comprises a plurality of pixels corresponding to one or more regions of the image.

Embodiment 30. The system of any one of embodiments 26-29, wherein the image comprises a histological stain image, a fluorescence in situ hybridization (FISH) image, an immunofluorescence (IF) image, or a hematoxylin and eosin (H&E) image.

Embodiment 31. The system of any one of embodiments 26-30, wherein:
the first layer comprises one or more convolutional layers;
the second layer comprises one or more batch normalization layers; and
the third layer comprises an output layer.

Embodiment 32. The system of embodiment 31, wherein the machine-learning model further comprises a pooling layer and a fully-connected layer.

Embodiment 33. The system of any one of embodiments 26-32, wherein the machine-learning model comprises one or more convolutional neural networks (CNNs).

Embodiment 34. The system of any one of embodiments 26-33, wherein the machine-learning model comprises a multiple-instance learning (MIL) machine-learning model.

Embodiment 35. The system of any one of embodiments 26-34, wherein the machine-learning model comprises a multiple-instance learning convolutional neural network (MILCNN) machine-learning model.

Embodiment 36. The system of any one of embodiments 26-35, wherein the set of batch normalization parameters comprises a mean and a variance determined from the at least one bag of patches.

Embodiment 37. The system of any one of embodiments 26-36, wherein the set of batch normalization parameters corresponds to only the at least one second bag of patches.

Embodiment 38. The system of any one of embodiments 26-37, wherein the machine-learning model was trained by:
receiving a training image;
segmenting the training image into a second plurality of patches;
grouping the second plurality of patches into at least one second bag of patches; and
inputting the at least one second bag of patches into the machine-learning model to generate a prediction of a second image class label based on the at least one second bag of patches;
wherein:
   the first layer is trained to generate one or more feature maps based on the at least one second bag of patches;
   the second layer is trained to normalize the one or more second feature maps utilizing a set of mini-batch normalization parameters determined from the at least one second bag of patches to generate one or more second normalized feature maps; and
   the third layer is trained to generate the prediction of the second image class label for the training image based at least in part on the one or more second normalized feature maps.

Embodiment 39. The system of embodiment 38, wherein each patch of the second plurality of patches comprises a plurality of pixels corresponding to one or more regions of the training image.

Embodiment 40. The system of embodiment 38 or 39, wherein:
the first layer comprises one or more convolutional layers;
the second layer comprises one or more batch normalization layers; and
the third layer comprises an output layer.

Embodiment 41. The system of embodiment 40, wherein the one or more batch normalization layers are trained to compute at least one of a running mean, a running variance, a gamma parameter, and a beta parameter of each of a plurality of sets of mini-batch normalization parameters during a training phase of the machine-learning model.

Embodiment 42. The system of embodiment 41, wherein, in response to the machine-learning model being trained, one or more of the gamma parameter and the beta parameter are fixed.

Embodiment 43. The system of any one of embodiments 38-42, wherein in response to the machine-learning model being trained, one or more of a mean and a variance determined for the at least one second bag of patches is configured to be determined for each additional bag of patches from the at least one second bag of patches.

Embodiment 44. The system of any one of embodiments 38-43, wherein the set of mini-batch normalization parameters comprises a mini-batch mean and a mini-batch variance.

Embodiment 45. The system of any one of embodiments 38-44, wherein the instructions to segment the training image into at least one second bag of patches further comprise instructions to randomly sampling one or more patches of pixels of the at least one second bag of patches.

Embodiment 46. The system of any one of embodiments 26-45, wherein the image class label comprises an indication of a genetic biomarker of a tissue sample captured in the image.

Embodiment 47. The system of embodiment 46, wherein the genetic biomarker of the tissue sample comprises an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, or one or more neurotrophic tyrosine receptor kinase (NTRK) genes 1/2/3.

Embodiment 48. The system of embodiment 46 or 47, wherein the instructions further comprise instructions generate a report based on the prediction of the image class label indicating the genetic biomarker of the tissue sample.

Embodiment 49. The system of embodiment 48, wherein the instructions further comprise instructions to cause one or more electronic devices to display the report.

Embodiment 50. The system of embodiment 49, wherein the instructions to cause the one or more electronic devices to display the report further comprise instructions to cause a human machine interface (HMI) associated with a pathologist to display the report.

Embodiment 51. A non-transitory computer-readable medium comprising instructions that, when executed by one or more processors of one or more computing devices, cause the one or more processors to:
segment, by the one or more processors, an image into a plurality of patches;
group, by the one or more processors, the plurality of patches into at least one bag of patches;
input, by the one or more processors, the at least one bag of patches into a machine-learning model trained to generate a prediction of an image class label based on the at least one bag of patches, the machine-learning model including:
   a first layer trained to generate one or more feature maps based on the at least one bag of patches;
   a second layer trained to normalize the one or more feature maps utilizing a set of batch normalization parameters determined from the at least one bag of patches to generate one or more normalized feature maps; and
   a third layer trained to generate the prediction of the image class label based at least in part on the one or more normalized feature maps; and
output, by the one or more processors, the prediction of the image class label.

Embodiment 52. The non-transitory computer-readable medium of embodiment 51, wherein the image comprises only one whole-slide image (WSI).

Embodiment 53. The non-transitory computer-readable medium of embodiment 51 or 52, wherein the instructions further comprise instructions to receive the image, wherein the image comprises an image of a tissue sample.

Embodiment 54. The non-transitory computer-readable medium of any one of embodiments 51-53, wherein each patch of the plurality of patches comprises a plurality of pixels corresponding to one or more regions of the image.

Embodiment 55. The non-transitory computer-readable medium of any one of embodiments 51-54, wherein the image comprises a histological stain image, a fluorescence in situ hybridization (FISH) image, an immunofluorescence (IF) image, or a hematoxylin and eosin (H&E) image.

Embodiment 56. The non-transitory computer-readable medium of any one of embodiments 51-55, wherein:
the first layer comprises one or more convolutional layers;
the second layer comprises one or more batch normalization layers; and
the third layer comprises an output layer.

Embodiment 57. The non-transitory computer-readable medium of any one of embodiments 51-56, wherein the machine-learning model further comprises a pooling layer and a fully connected layer.

Embodiment 58. The non-transitory computer-readable medium of any one of embodiments 51-57, wherein the machine-learning model comprises one or more convolutional neural networks (CNNs).

Embodiment 59. The non-transitory computer-readable medium of any one of embodiments 51-58, wherein the machine-learning model comprises a multiple-instance learning (MIL) machine-learning model.

Embodiment 60. The non-transitory computer-readable medium of any one of embodiments 51-59, wherein the machine-learning model comprises a multiple-instance learning convolutional neural network (MILCNN) machine-learning model.

Embodiment 61. The non-transitory computer-readable medium of any one of embodiments 51-60, wherein the set of batch normalization parameters comprises a mean and a variance determined from the at least one bag of patches.

Embodiment 62. The non-transitory computer-readable medium of any one of embodiments 51-61, wherein the set of batch normalization parameters corresponds to only the at least one second bag of patches.

Embodiment 63. The non-transitory computer-readable medium of any one of embodiments 51-62, wherein the machine-learning model was trained by:
receiving a training image;
segmenting the training image into a second plurality of patches;
grouping the second plurality of patches into at least one second bag of patches; and
inputting the at least one second bag of patches into the machine-learning model to generate a prediction of a second image class label based on the at least one second bag of patches;
wherein:
   the first layer is trained to generate one or more feature maps based on the at least one second bag of patches;
   the second layer is trained to normalize the one or more second feature maps utilizing a set of mini-batch normalization parameters determined from the at least one second bag of patches to generate one or more second normalized feature maps; and
   the third layer is trained to generate the prediction of the second image class label for the training image based at least in part on the one or more second normalized feature maps.

Embodiment 64. The non-transitory computer-readable medium of embodiment 63, wherein each patch of the second plurality of patches comprises a plurality of pixels corresponding to one or more regions of the training image.

Embodiment 65. The non-transitory computer-readable medium of embodiment 63 or 64, wherein:
the first layer comprises one or more convolutional layers;
the second layer comprises one or more batch normalization layers; and
the third layer comprises an output layer.

Embodiment 66. The non-transitory computer-readable medium of embodiment 65, wherein the one or more batch normalization layers are trained to compute at least one of a running mean, a running variance, a gamma parameter, and a beta parameter of each of a plurality of sets of mini-batch normalization parameters during a training phase of the machine-learning model.

Embodiment 67. The non-transitory computer-readable medium of embodiment 66, wherein, in response to the machine-learning model being trained, one or more of the gamma parameter and the beta parameter are fixed.

Embodiment 68. The non-transitory computer-readable medium of any one of embodiments 65-67, wherein in response to the machine-learning model being trained, one or more of a mean and a variance determined for the at least one second bag of patches is configured to be determined for each additional bag of patches from the at least one second bag of patches.

Embodiment 69. The non-transitory computer-readable medium of any one of embodiments 65-68, wherein the set of mini-batch normalization parameters comprises a mini-batch mean and a mini-batch variance.

Embodiment 70. The non-transitory computer-readable medium of any one of embodiments 65-69, wherein the instructions to segment the training image into at least one second bag of patches further comprise instructions to randomly sampling one or more patches of pixels of the at least one second bag of patches.

Embodiment 71. The non-transitory computer-readable medium of any one of embodiments 51-70, wherein the image class label comprises an indication of a genetic biomarker of a tissue sample captured in the image.

Embodiment 72. The non-transitory computer-readable medium of embodiment 71, wherein the genetic biomarker of the tissue sample comprises an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, or one or more neurotrophic tyrosine receptor kinase (NTRK) genes 1/2/3.

Embodiment 73. The non-transitory computer-readable medium of embodiment 71 or 72, wherein the instructions further comprise instructions generate a report based on the prediction of the image class label indicating the genetic biomarker of the tissue sample.

Embodiment 74. The non-transitory computer-readable medium of embodiment 73, wherein the instructions further comprise instructions to cause one or more electronic devices to display the report.

Embodiment 75. The non-transitory computer-readable medium of embodiment 74, wherein the instructions to cause the one or more electronic devices to display the report further comprise instructions to cause a human machine interface (HMI) associated with a pathologist to display the report.

Embodiment 76. A method, comprising:
receiving, by one or more processors, a training image;
segmenting, by the one or more processors, the training image into a plurality of patches;
grouping, by the one or more processors, the plurality of patches into at least one bag of patches;
training a first layer to generate one or more feature maps based on the at least one bag of patches;
training a second layer to normalize the one or more feature maps utilizing a set of mini-batch normalization parameters from the one or more normalized feature maps; and
training a third layer to generate the prediction of an image class label for the training image based at least in part on the one or more normalized feature maps.

Embodiment 77. The method of embodiment 76, wherein the training image is a single image.

Embodiment 78. The method of embodiment 76 or 77, wherein each patch of the plurality of patches comprises a plurality of pixels corresponding to one or more regions of the training image.

Embodiment 79. The method of any one of embodiments 76-78, wherein:
the first layer comprises one or more convolutional layers;
the second layer comprises one or more batch normalization layers; and
the third layer comprises an output layer.

Embodiment 80. The method of embodiment 79, wherein the one or more batch normalization layers are trained to compute at least one of a running mean, a running variance, a gamma parameter, and a beta parameter of each of a plurality of sets of mini-batch normalization parameters during a training phase.

Embodiment 81. The method of embodiment 80, wherein the set of mini-batch normalization parameters comprises a mini-batch mean and a mini-batch variance.

Embodiment 82. The method of any one of embodiments 76-81, wherein segmenting the training image into at least one bag of patches comprises randomly sampling one or more patches of pixels of the at least one bag of patches.

Embodiment 83. A method, comprising:
receiving, by one or more processors, an image of a tissue sample;
segmenting, by the one or more processors, the image into a plurality of bags of patches, wherein each patch of the plurality of bags of patches comprises a plurality of pixels corresponding to one or more regions of the tissue sample;
inputting, by the one or more processors, at least one bag of patches of the plurality of bags of patches into a machine-learning model trained to generate a prediction of an image class label indicating a genetic biomarker of the tissue sample based on the at least one bag of patches, the machine-learning model including:
   first layer trained to generate one or more feature maps based on the at least one bag of patches;
   a second layer trained to normalize the one or more feature maps utilizing a set of batch normalization parameters determined from the at least one bag of patches to generate one or more normalized feature maps; and
   a third layer trained to generate the prediction of the image class label indicating a genetic biomarker of the tissue sample based at least in part on the one or more normalized feature maps; and
outputting, by the one or more processors, the prediction of the image class label indicating a genetic biomarker of the tissue sample.

Embodiment 84. The method of embodiment 83, wherein the image of the tissue sample comprises only one whole-slide image (WSI) of one or more cancer tissue samples.

Embodiment 85. The method of embodiment 83 or 84, wherein the image of the tissue sample comprises a histological stain image of the one or more cancer tissue samples, a fluorescence in situ hybridization (FISH) image of the one or more cancer tissue samples, an immunofluorescence (IF) image of the one or more cancer tissue samples, or a hematoxylin and eosin (H&E) image of the one or more cancer tissue samples.

Embodiment 86. The method of any one of embodiments 83-85, wherein:
the first layer comprises one or more convolutional layers;
the second layer comprises one or more batch normalization layers; and
the third layer comprises an output layer.

Embodiment 87. The method of embodiment 86, wherein the machine-learning model further comprises a pooling layer and a fully connected layer.

Embodiment 88. The method of any one of embodiments 83-87, wherein the set of batch normalization parameters comprises a mean and a variance determined from the at least one bag of patches.

Embodiment 89. The method of any one of embodiments 83-88, wherein the set of batch normalization parameters corresponds to only the at least one second bag of patches.

Embodiment 90. The method of any one of embodiments 83-89, wherein the machine-learning model was trained by:
receiving, by the one or more processors, a training image;
segmenting, by the one or more processors, the training image into a second plurality of patches;
grouping, by the one or more processors, the second plurality of patches into at least one second bag of patches; and
inputting, by the one or more processors, the at least one second bag of patches into the machine-learning model to generate a prediction of a second image class label based on the at least one second bag of patches;
wherein:
   the first layer is trained to generate one or more feature maps based on the at least one second bag of patches;
   the second layer is trained to normalize the one or more second feature maps utilizing a set of mini-batch normalization parameters determined from the at least one second bag of patches to generate one or more second normalized feature maps; and
   the third layer is trained to generate the prediction of the second image class label for the training image based at least in part on the one or more second normalized feature maps.

Embodiment 91. The method of embodiment 90, wherein each patch of the second plurality of patches comprises a plurality of pixels corresponding to one or more regions of the training image.

Embodiment 92. The method of embodiment 90 or 91, wherein:
the first layer comprises one or more convolutional layers;
the second layer comprises one or more batch normalization layers; and
the third layer comprises an output layer.

Embodiment 93. The method of embodiment 92, wherein the one or more batch normalization layers are trained to compute at least one of a running mean, a running variance, a gamma parameter, and a beta parameter of each of a plurality of sets of mini-batch normalization parameters during a training phase of the machine-learning model.

Embodiment 94. The method of embodiment 93, wherein, in response to the machine-learning model being trained, one or more of the gamma parameter and the beta parameter are fixed.

Embodiment 95. The method of any one of embodiments 90-94, wherein in response to the machine-learning model being trained, one or more of a mean and a variance determined for the at least one second bag of patches is configured to be determined for each additional bag of patches from the at least one second bag of patches.

Embodiment 96. The method of any one of embodiments 90-95, wherein the set of mini-batch normalization parameters comprises a mini-batch mean and a mini-batch variance.

Embodiment 97. The method of any one of embodiments 90-96, wherein segmenting the training image into at least one second bag of patches comprises randomly sampling one or more patches of pixels of the at least one second bag of patches.

Embodiment 98. The method of any one of embodiments 90-97, further comprising generating a report based on the prediction of the image class label indicating the genetic biomarker of the tissue sample.

Embodiment 99. The method of embodiment 98, further comprising causing one or more electronic devices to display the report.

Embodiment 100. The method of embodiment 99, wherein causing the one or more electronic devices to display the report comprises causing a human machine interface (HMI) associated with a pathologist to display the report.

Embodiment 101. The method of any one of embodiments 76-100, wherein the machine-learning model comprises one or more convolutional neural networks (CNNs).

Embodiment 102. The method of any one of embodiments 76-101, wherein the machine-learning model comprises a multiple-instance learning (MIL) machine-learning model.

Embodiment 103. The method of any one of embodiments 76-102, wherein the machine-learning model comprises a multiple-instance learning convolutional neural network (MILCNN) machine-learning model.

Embodiment 104. The method of any one of embodiments 76-103, wherein the image class label comprises an indication of a genetic biomarker of a tissue sample captured in the image.

Embodiment 105. The method of embodiment 104, wherein the genetic biomarker of the tissue sample comprises an epidermal growth factor receptor (EFGR) gene alteration, an anaplastic lymphoma kinase (ALK) gene alteration, an ROS-1 gene alteration, a tumor gene mutation burden (TMB), neurotrophic tyrosine receptor kinase 3 (NTRK3) gene alteration, a fibroblast growth factor receptor 2 (FGFR2) gene alteration, mesenchymal-epithelial transition (MET) gene alteration, phosphatidylinositol-4,5-bisphosphate 3-Kinase catalytic subunit alpha (PIK3CA) gene alteration, or one or more neurotrophic tyrosine receptor kinase (NTRK) genes 1/2/3.

### Example

The present disclosure will be more fully understood by reference to the following Example. It should not, however, be construed as limiting the scope of the present disclosure.

Treatment of non-small cell lung cancer is increasingly biomarker driven with multiple genomic alterations, including those in the epidermal growth factor receptor (EGFR) gene, that benefit from targeted therapies. As further described, an algorithm was developed to assess EGFR status and morphology using a real-world advanced lung adenocarcinoma cohort of 2099 patients with hematoxylin and eosin (H&E) images exhibiting high morphological diversity and low tumor content relative to public datasets. The attention-based EGFR algorithm achieved an area under the curve (AUC) of 0.870, a negative predictive value (NPV) of 0.954 and a positive predictive value (PPV) of 0.410 in a validation cohort reflecting the 15% prevalence of EGFR mutations in lung adenocarcinoma. The attention model outperformed a heuristic-based model focused exclusively on tumor regions used as a comparison. Although the attention model also extracts signal primarily from tumor morphology, it extracts additional signal from non-tumor tissue regions. Further analysis of high-attention regions by pathologists showed associations of predicted EGFR negativity with solid growth patterns and higher peritumoral immune presence. This algorithm highlights the potential of this process to provide instantaneous rule-out screening for biomarker alterations and may help prioritize the use of scarce tissue for biomarker testing. Although this Example was trained to call EGFR statuses, similar models may be trained to call the mutational status of different genes, such as ALK, ROS1, FGFR2, MET, PIK3CA, NTRK1, NTRK2, or NTRK3.

As further discussed, attention-based multiple-instance learning can predict EGFR mutational status in advanced metastatic lung adenocarcinoma samples directly from H&E images with state-of-the-art performance on real-world datasets, where many samples have less than 50% tumor content. Through a combination of tissue morphology classification models and pathologist review it is shown that although tumor regions contain the most signal for EGFR, the attention-based model also considers relevant outlier instances from other tissue types such as immune or stromal features when predicting EGFR mutational status. With additional analysis via association rules mining, a process is demonstrated wherein morphology models and pathologist expertise can be leveraged to biologically verify end-to-end biomarker predictions by evaluating associated feature combinations, allowing for better model interpretation when supporting clinical decisions.

### Methods

Dataset. The dataset used in this study consisted of lung adenocarcinoma resection H&E whole slide image scans acquired from specimens submitted to for genomic profiling. All images within this dataset were scanned at 20× magnification. This image dataset was generated from 2099 tissue specimens from 2099 individual patients. 716 of the specimens were determined by genomic sequencing to be EGFR short-variant mutant specimens. Of the remaining specimens, 85 were ALK mutated, 93 BRAF mutated, 81 ERBB2 mutated, 606 KRAS mutated, 76 MET mutated, 35 RET mutated, 18 ROS1 mutated, and 389 were lung driver wild-type.

Five-fold cross-validation was performed to evaluate model performance and consistency. For ground-truth, all slides used the specimen-level mutational statuses as determined by a next-generation sequencing test. The training/validation split for all experiments was 0.8/0.2 for EGFR mutant slides. The real-world prevalence of EGFR short variant mutations is approximately 15% in NSCLC, and thus represents a minority class for which class-imbalanced modeling was a consideration. As the available data contained a relatively large number of EGFR mutated lung adenocarcinoma specimens, minority class balancing techniques such as minority over-sampling or minority class weight penalization were not applied. Instead, majority under-sampling was performed, randomly selecting an equal number of slides that were not EGFR mutated to balance the EGFR mutated slides in the training set. For the validation sets, enough slides were selected that were not EGFR mutated so that the percentage of EGFR mutated slides in the validation was 15%, reflecting the real-world prevalence. This aimed to simplify the training process while still allowing for an evaluation of the model against a validation dataset that more closely represented a real-world setting. As a result, each training set had 1146 slides and each validation set had 953 slides.

Model Architecture. The attention-based multiple-instance learning model was built using ResNet50 without the top-layer and with an added global average pooling layer to serve as a trainable feature extractor. Following the feature extractor was an attention-mechanism including two fully-connected layers (512-dimensional, 256-dimensional) to reduce the embedding dimensionality. The reduced embeddings were then passed to a 256-dimensional fully-connected layer followed by another 1-dimensional fully-connected layer. The output is then transposed and all patches within a multiple-instance bag are passed through a softmax activation which fractionally weighs the attention for each patch within the bag. The reduced embeddings are then weighed using the softmax attention weights to generate the slide-level weighted embedding. A final fully-connected layer processes the slide-level weighted embedding and uses the sigmoid activation to predict the specimen-level EGFR status.

Training procedure. For the training inputs of the EGFR mutation prediction model, raw 1024×1024 pixel patches were resized to 224×224 pixel patches to allow each bag in the MIL formulation to include more patches, as limited by GPU memory, so that the MIL model would be allowed a more holistic view of each slide.

The EGFR mutation prediction task is a binary classification task where, given a bag of patches (where the bag as a whole has a label but patches individually do not), a prediction from an H&E image was made as to whether a gene mutation is present within a specimen. The final layer for the model is a fully-connected with a one-dimensional output and the activation function used is the sigmoid function:

$$g ( z ) = \frac{1}{1 + {e}^{- z}}$$

$$where$$

$$z = {w}^{T} x + b$$

and where x is the input to the fully-connected output layer, w is the weight matrix of the output layer, and b is the bias term for the output layer. The loss is optimized for is the binary cross-entropy loss:

$$l = - ( \hat{y} log ( g ( z ) ) + ( 1 - \hat{y} ) log ( 1 - g ( z ) )$$

where ŷ is the target value for the input sample. The batch loss is aggregated across each input sample within the batch by either summing or averaging the losses, and gradient descent is performed to update the model parameters. The MIL models were trained for 200 epochs with 40 patches per bag during each training pass, trained using the TensorFlow³³ framework. The Adam³⁴ optimizer was used with a learning rate of 1e-5.

Additionally, when running inference on the validation slides, it was found that performance was notably better if batch normalization layers used batch statistics for normalization instead of using the exponentially decaying running mean and variance tracked during training. As each training step involved processing bags from at most one or two slides due to GPU memory constraints, it was found that generalizability to the validation set suffered if using the standard momentum-based training statistics for batch normalization, as each batch processed would not be a sampling from the overall cohort population but rather from a very limited number of slides. If each slide during validation is processed individually, then the patch instances within each batch are drawn from the same slide and thus the instance interdependence in the batch formulation is non-arbitrary. This is analogous to vision applications extracting multiple regions-of-interest from a single image and composing those regions-of-interest into a batch to utilize batch statistics for inference.

Pathologist review of high-attention patches from high-confidence bags. To examine the attention learned by our MIL models and to better understand what features were relevant for predicting EGFR mutant versus wild-type specimens, expert pathologists evaluated high-attention patches for bags confidently predicted to be mutant or wild-type. For 49 validation slides, 250 patches per bag were sampled and each bag was passed through the trained MIL models. The patches within each bag were then ordered by descending attention weight. The top-25 highest-attention patches for each of the 49 bags were provided to pathologists for analysis, resulting in a total of 1225 patches being reviewed.

Pathologists scored each patch for a set of numerical variables and then further reviewed each patch for categorical characteristics. The numerical variables were tumor nuclei fraction, necrosis fraction, peritumoral immune fraction, and intratumoral immune fraction. Tumor nuclei fraction was determined as the fraction of tumor nuclei relative to all nuclei present within a patch. Necrosis fraction was determined as the fraction of the patch area containing necrotic tissue. The peritumoral immune fraction was determined as the fraction of tumor edges that had noticeable immune cell response, such as lymphocytes aggregating at or within the tumor boundary. The intratumoral immune fraction was determined as the fraction of tumor tissue within a patch that had noticeable immune infiltration, such as lymphocytes dispersed throughout a tumor mass or nest.

For the review of categorical variables, pathologists examined each patch for the tumor's predominant architectural pattern, minor architectural pattern, cytology, and any notable non-neoplastic quality. The possible predominant and minor architectural patterns were acinar, lepidic, papillary, micropapillary, mucinous, and solid. The possible cytology types were hobnail, columnar, mucinous, sarcomatoid, anaplastic, large cell, small cell, or other. Non-neoplastic qualities included fibrosis, pneumonia, inflammation, or other.

In order to evaluate overall bag characteristics relative to the model's mutant predictions versus wild-type predictions, summary statistics and overall characteristics from the pathologist review of the high-attention patches were generated. To determine each bag's overall numerical statistics, the mean, standard deviation, minimum, and maximum of the numerical scores provided by pathologists across the top-25 high attention patches from that bag were calculated. To determine the bag's overall categorical characteristics, the patch reviews across the top-attention patches was aggregated by taking the mode. Thus, each bag had an overall summary of patch scores and categorical labels for the high-attention patches, which could then be compared based on the model's predicted EGFR mutation status.

Significance between the predicted mutant and predicted wild-type slides with respect to the numerical variables was tested using the two-way T-test. False discovery rate correction was additionally applied to generate q-values from T-test p-values. No comparisons were significant after false discovery rate correction. Categorical comparisons were completed using the Chi-square test, at an overall bag level. Finally, association rules mining was performed by treating each overall categorical value determined for the bags as items, with predicted EGFR status as the consequent item-set.

### Results

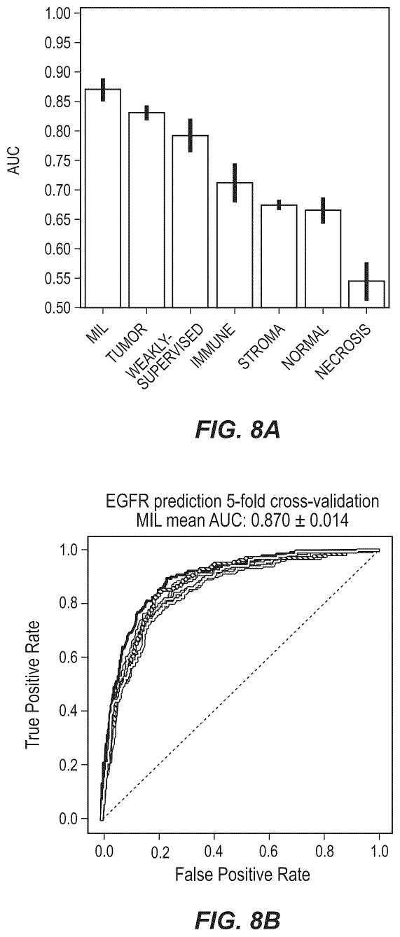

Bags of patches were randomly sampled from each slide during training and the entire bag was given the specimen-level EGFR status as the label. Through the attention mechanism, the model learned without human guidance how to weigh different patches within each bag when predicting for specimen-level mutational status. The AUC achieved by the MIL models with five-fold cross-validation was 0.870±0.014, which was significantly higher than a comparator tumor-only models (FIG. 8A and FIG. 8B; p=0.002). The models also achieved an NPV of 0.954±0.024 and a PPV of 0.41±0.081 at a binary classification threshold of 0.5. If only slides with high-confidence predictions (defined as <0.25 for wild-type call and >0.75 for mutant call) were considered, the NPV was 0.970±0.017 and the PPV was 0.527±0.088. Thus, attention-based models outperformed the comparator human-guided tumor-only models.

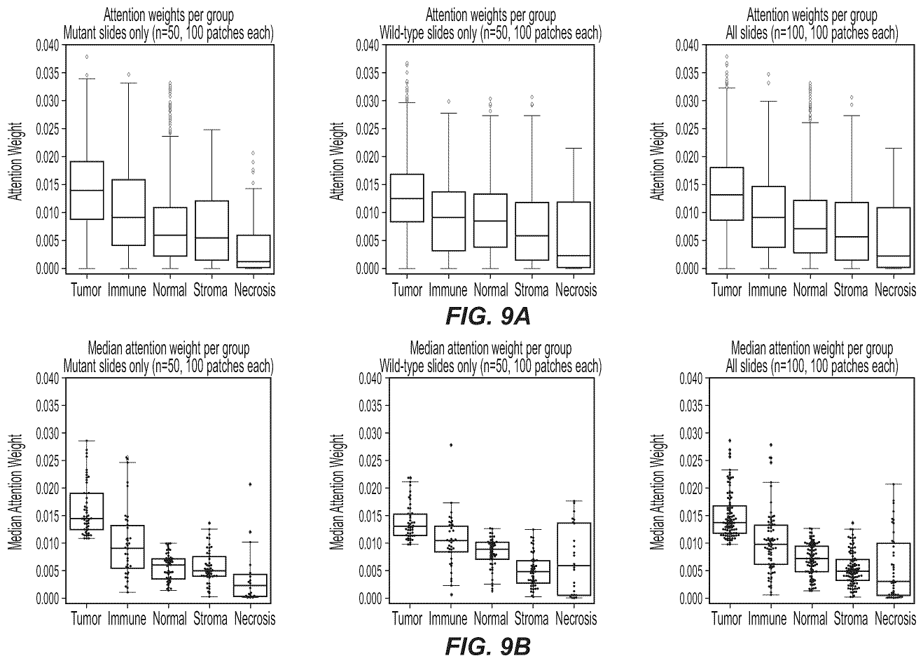

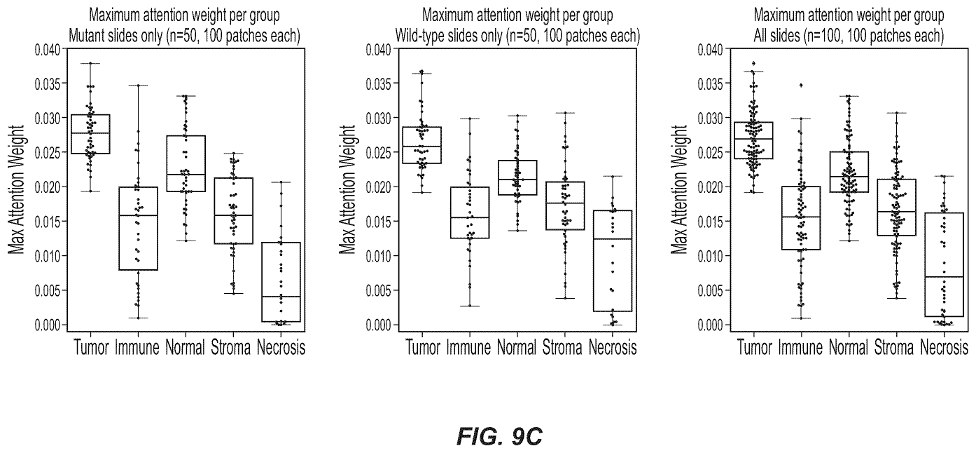

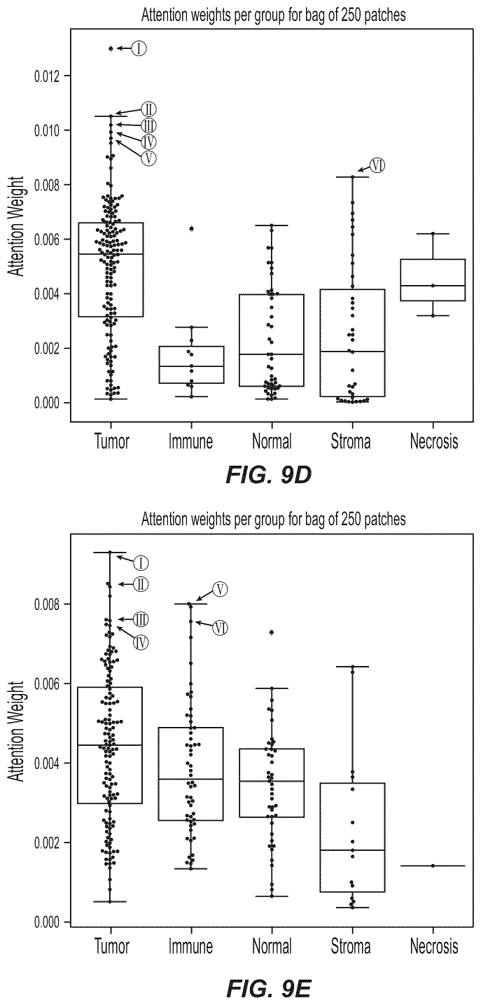

To better understand what features the MIL models might have learned, it was then investigated how the MIL models distributed their attention across patch morphologies. To do this, 100 patches per bag were sampled for 100 validation slides and passed through the MIL models to assess attention by tissue morphology (FIGS. 9A-9C). We found that the median attention score was highest for tumor patches at 0.013 with a maximum score of 0.038. As a group, the immune patches were second with a median attention score of 0.009 and a maximum of 0.035. The median attention given to normal patches, stroma patches, and necrosis patches were 0.007, 0.006, and 0.002 with corresponding maximum attention scores of 0.033, 0.031, and 0.022, respectively. The tissue morphology classification of patches also allowed pathologists to quickly assess any high-attention outlier patches, especially those belonging to the non-tumor groups, for noteworthy visual features, as shown in FIGS. 9D and 9E. In FIG. 9D, an EGFR true positive (TP) exemplar is presented. High attention was given to tumor and stroma patches. Patches I-V had a predominant acinar pattern and hobnail cytology, with low peritumoral and intratumoral immune fractions, ranging from 0.1 to 0.2. Patch IV had a low presence of necrotic tissue and patch VI was predicted as stroma by the tissue-morphology model, and pathologists confirmed this patch was fibrosis. In FIG. 9E, an EGFR true negative (TN) exemplar is presented. High attention was given to tumor patches and some immune patches. Patches I-II showed an acinar/lepidic pattern with hobnail cytology and intratumoral lymphoid aggregates. Patches III-VI were predicted to be tumor or immune foci by the tissue-morphology model. Pathologists confirmed high peritumoral and intratumoral immune fraction, ranging from 0.2 to 0.7, for these patches. Inflammation was noticeably present as well in patch IV. From these data we conclude that the MIL models learned to give high attention to tumor regions but likely boosted performance by also giving high attention to additional patterns that aid in classification such as immune infiltrates in EGFR negative samples.

To investigate the learned-attention of the models at a more detailed level, pathologists reviewed the top-25 highest attention patches for each of 49 randomly sampled bags for which the MIL models produced high confidence predictions. When the MIL models predicted for EGFR mutational status, there was a significant difference when considering the standard deviation of tumor nuclei fraction across the highest-attention patches for each bag, which was lower for bags predicted as EGFR mutant (p=0.028, Pearson's r: −0.317). The minimum tumor nuclei fraction across the high-attention patches was significantly higher for bags predicted to be EGFR mutant (p=0.037, Pearson's r: 0.301). The maximum peritumoral immune fraction across high-attention patches was significantly lower for bags predicted to be EGFR mutant (p=0.041, Pearson's r: −0.297).

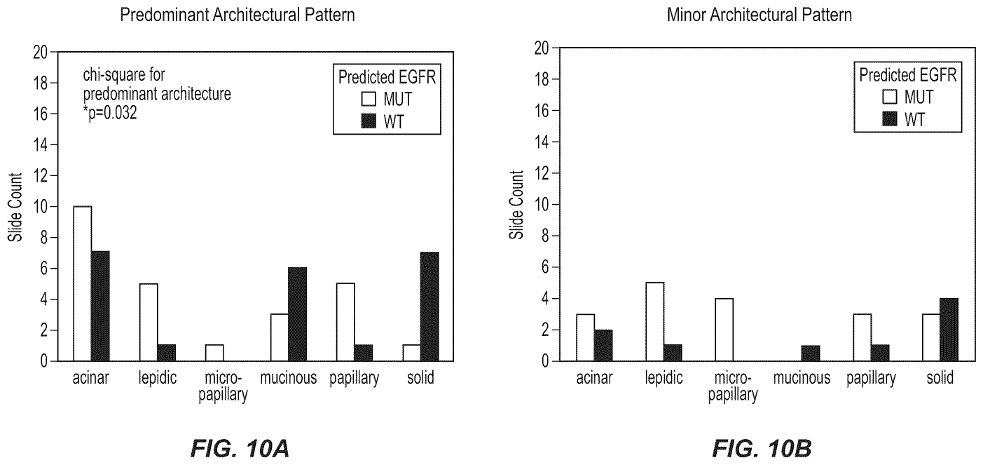

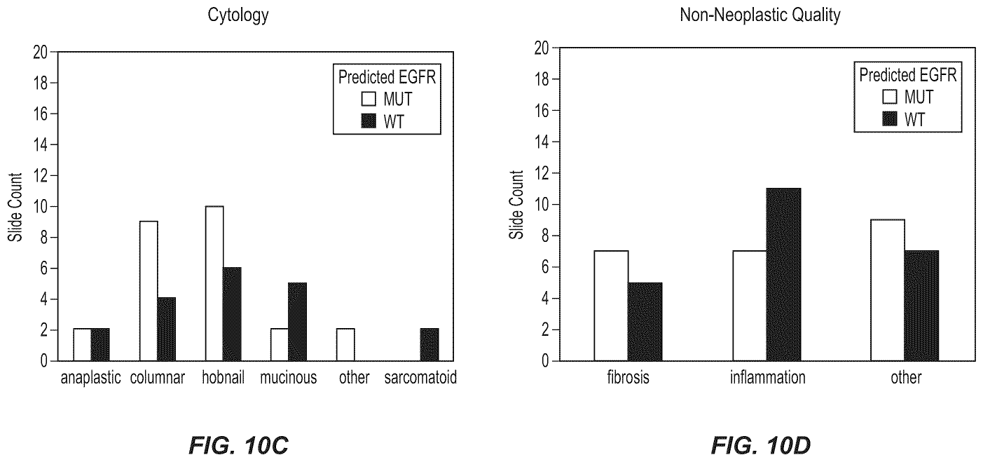

Chi-squared testing for each bag's predicted status and the overall predominant architectural pattern, as determined by the mode of the bag's patch characterizations, showed significance (FIG. 10A; p=0.035) when predicting EGFR mutations versus wild-type. There were significantly more bags predicted wild-type than EGFR mutant when the predominant architectural pattern was solid (p=0.013, value not shown in FIG. 10A).

To investigate categorical patterns learned by the models, each bag's overall characteristics were summarized by determining the mode of each category for the bag's reviewed patches. When considering overall architecture, bags that were predominantly lepidic or papillary were predicted EGFR mutant five times more often than EGFR wild-type (FIG. 10A). On the contrary, bags that predominantly possessed the solid architecture were predicted as EGFR wild-type seven times more often than mutant. When the predominant architecture was mucinous, it was twice as likely that the bag would be predicted as EGFR wild-type. There was no strong enrichment (ratio <2.0) in prediction status of either type for predominantly acinar bags. All bags with any micropapillary content (predominant or minor) were predicted as EGFR mutant specimens (FIG. 10A and FIG. 10B). The directionality of preference for predicted status when considering acinar, lepidic, papillary, mucinous for the minor architectures were similar to the preference in the predominant architecture, but the solid minor architectural pattern did not see the same strength of preference for EGFR mutant predictions compared to instances where the solid architecture was the predominant pattern. From a cytology perspective, bags with columnar or hobnail as the most common cell type across the high-attention patches were more likely (>1.5) to be predicted as mutant (FIG. 10C). Mucinous and sarcomatoid cytologies were more likely to be predicted as wild-type. From an overall tumor-feature perspective, our MIL models tended to predict lepidic and papillary patterns as EGFR mutant and any mucinous characteristic (architecture and cytology) as EGFR wild-type (FIG. 10C). For non-neoplastic qualities, slides with inflammation were more likely to be predicted as EGFR wild-type (FIG. 10D). Generally, there were no categorical characteristics (aside from the micropapillary pattern) that perfectly separated specimens by predicted status, possibly suggesting that the models consider the various characteristics within each bag in combination.

To examine the relevance of the patch characterization in a combinatorial manner, we performed association rules mining (see Agrawal et al., Mining Association Rules Between Sets of Items in Large Databases, ACM SIGMOD, Record 22, (1993)) to determine item-sets of interest using the categorical variables. Each bag's overall characterization was determined via the category mode for the reviewed patches in the bag. The highest-lift item-sets for predicted wild-type status as a consequent included: {inflammation, hobnail cytology, solid minor architectural pattern}, {inflammation, acinar predominant architectural pattern, hobnail cytology}, {acinar predominant architectural pattern, hobnail cytology, solid minor architectural pattern} and {acinar predominant architectural pattern, inflammation, hobnail cytology, solid minor architectural pattern}, each with a lift of 2.097. In contrast, the highest-lift item-sets for predicted EGFR mutated status included: {fibrosis, lepidic minor architectural pattern, hobnail cytology} and {fibrosis, acinar predominant architectural pattern, hobnail cytology}, both with a lift of 1.92. In total, the EGFR prediction algorithm recapitulates several known morphological and cytological associations with EGFR status and these features can be tested on a per sample basis by analyzing highly attended regions manually or via tissue morphology/cytology classification algorithms.

### Discussion

As barriers to clinical adoption of digital tools are reduced, the development of machine learning models to augment and support established processes is highly desirable. However, models trained on research datasets that are dissimilar to real-world data may have difficulty generalizing in a clinical setting, where the incoming sample distribution may not align well with the training data. With this in mind, described herein is a machine learning model that predicts EGFR mutational status on real-world H&E lung adenocarcinoma images with high morphological diversity and shows the potential for use as screening algorithms with high NPV. It was demonstrated that state-of-the-art performance for predicting EGFR can be achieved by using attention-based models that evaluate a full range of tissue morphologies, outperforming other models. Additionally, attention-based models do not require expensive manual annotation or guidance to train. Finally, it was shown that biological verification of attention-based end-to-end models can be performed by combining assessment approaches such as morphological profiling, item-set analysis, and pathology review, potentially increasing accuracy in a clinical setting.

Beyond tumor-associated features, it has also been suggested that immune response and non-neoplastic components within the tumor microenvironment (TME) may be relevant when examining the effect of mutations upon linked biological pathways. The MIL model described herein appears to learn the trend of lowered immune response within the TME of EGFR mutated specimens, in part indicated by the significantly higher maximum peritumoral immune fraction (p=0.041) across high-attention patches for specimens strongly predicted to be wild-type. Additionally, inflammation is present within three out of four of the highest-lift item-sets for EGFR wild-type predictions, while it is absent from the highest-lift item-sets for EGFR mutant predictions.

Finally, the ability to examine the attention given by MIL models may allow exploration of other less obvious elements within the TME that could help elucidate the biological understanding of EGFR mutations. In two of the highest-lift item-sets for predicted EGFR mutant status, fibrosis is present alongside the tumor-related features. This inclusion of fibrosis is less expected than the inclusion of tumor features but may also suggest interesting interactions within the TME. Many studies now suggest that stroma and stromal elements may play far more than a passive role within TMEs and may have direct effects on tumorigenesis. The inclusion of fibrosis as a relevant feature may indicate the ability of machine learning models to recognize, without human guidance, patterns involving tissue regions that may be orthogonal to tumor-specific features.

The machine learning model enabled with self-directed intuition such as attention-based MIL models can predict EGFR mutational status from morphologically-diverse real-world tissue specimens without human intervention. The ability to rely upon machine-intuition to extract meaningful features could enable low-effort signal-searching experiments at scale, as well as provide a means to investigate machine-discovered patterns within the phenotype that may be biologically informative. It is encouraging from an interpretability standpoint that models intended to assist in clinical decision-making recapitulate expected results, such as finding tumor regions most predictive for genomic alteration signal, but also that such models may be capable of determining patterns and interactions within phenotypic features in ways that elevate performance beyond methods relying solely upon human intuition. In a clinical setting, these screening algorithms could provide rapid genomic insights regarding a patient specimen, which can then be checked by a combination of more interpretable models as well as pathologist visual examination. Any low-confidence predictions or samples flagged by pathologists could then be selected for further genomic testing.

1. A method, comprising:
segmenting, by one or more processors, an image into a plurality of patches;
grouping, by the one or more processors, the plurality of patches into at least one bag of patches;
inputting, by the one or more processors, the at least one bag of patches into a machine-learning model trained to generate a prediction of an image class label based on the at least one bag of patches, the machine-learning model including:
a first layer trained to generate one or more feature maps based on the at least one bag of patches;
a second layer trained to normalize the one or more feature maps utilizing a set of batch normalization parameters determined from the at least one bag of patches to generate one or more normalized feature maps; and
a third layer trained to generate the prediction of the image class label based at least in part on the one or more normalized feature maps; and
outputting, by the one or more processors, the prediction of the image class label.

2. The method of claim 1, wherein the image comprises only one whole-slide image (WSI).

3. The method of claim 1, further comprising receiving, by the one or more processors, the image, wherein the image comprises an image of a tissue sample.

4. The method of claim 1, wherein each patch of the plurality of patches comprises a plurality of pixels corresponding to one or more regions of the image.

5. The method of claim 1, wherein the image comprises a histological stain image, a fluorescence in situ hybridization (FISH) image, an immunofluorescence (IF) image, or a hematoxylin and eosin (H&E) image.

6. The method of claim 1, wherein:
the first layer comprises one or more convolutional layers;
the second layer comprises one or more batch normalization layers; and
the third layer comprises an output layer.

7. The method of claim 6, wherein the machine-learning model further comprises a pooling layer and a fully connected layer.

8. The method of claim 1, wherein the machine-learning model comprises one or more convolutional neural networks (CNNs), a multiple-instance learning (MIL) machine-learning model, or a multiple-instance learning convolutional neural network (MILCNN) machine-learning model.

9-10. (canceled)

11. The method of claim 1, wherein the set of batch normalization parameters comprises a mean and a variance determined from the at least one bag of patches.

12. The method of claim 1, wherein the set of batch normalization parameters corresponds to only the at least one second bag of patches.

13. The method of claim 1, wherein the machine-learning model was trained by:
receiving, by the one or more processors, a training image;
segmenting, by the one or more processors, the training image into a second plurality of patches;
grouping, by the one or more processors, the second plurality of patches into at least one second bag of patches; and
inputting, by the one or more processors, the at least one second bag of patches into the machine-learning model to generate a prediction of a second image class label based on the at least one second bag of patches;
wherein:
the first layer is trained to generate one or more feature maps based on the at least one second bag of patches;
the second layer is trained to normalize the one or more second feature maps utilizing a set of mini-batch normalization parameters determined from the at least one second bag of patches to generate one or more second normalized feature maps; and
the third layer is trained to generate the prediction of the second image class label for the training image based at least in part on the one or more second normalized feature maps.

14. The method of claim 13, wherein each patch of the second plurality of patches comprises a plurality of pixels corresponding to one or more regions of the training image.

15. The method of claim 13, wherein:
the first layer comprises one or more convolutional layers;
the second layer comprises one or more batch normalization layers; and
the third layer comprises an output layer.

16. The method of claim 15, wherein the one or more batch normalization layers are trained to compute at least one of a running mean, a running variance, a gamma parameter, and a beta parameter of each of a plurality of sets of mini-batch normalization parameters during a training phase of the machine-learning model.

17-18. (canceled)

19. The method of claim 13, wherein the set of mini-batch normalization parameters comprises a mini-batch mean and a mini-batch variance.

20. The method of claim 13, wherein segmenting the training image into at least one second bag of patches comprises randomly sampling one or more patches of pixels of the at least one second bag of patches.

21. The method of claim 1, wherein the image class label comprises an indication of a genetic biomarker of a tissue sample captured in the image.

22-25. (canceled)

26. A method of treating subject with cancer, comprising:
characterizing a tissue sample comprising the cancer from the subject as having a genetic biomarker according to the method of claim 21; and
administering to the subject an effect treatment for the cancer based on the tissue sample having the genetic biomarker.

27. A system including one or more computing devices, comprising:
one or more non-transitory computer-readable storage media including instructions; and
one or more processors coupled to the one or more storage media, the one or more processors configured to execute the instructions to:
segment an image into a plurality of patches;
group the plurality of patches into at least one bag of patches; and
input the at least one bag of patches into a machine-learning model trained to generate a prediction of an image class label based on the at least one bag of patches, the machine-learning model including:
a first layer trained to generate one or more feature maps based on the at least one bag of patches;
a second layer trained to normalize the one or more feature maps utilizing a set of batch normalization parameters determined from the at least one bag of patches to generate one or more normalized feature maps; and
a third layer trained to generate the prediction of the image class label based at least in part on the one or more normalized feature maps; and
output the prediction of the image class label.

28. (canceled)

29. A method, comprising:
receiving, by one or more processors, a training image;
segmenting, by the one or more processors, the training image into a plurality of patches;
grouping, by the one or more processors, the plurality of patches into at least one bag of patches;
training a first layer to generate one or more feature maps based on the at least one bag of patches;
training a second layer to normalize the one or more feature maps utilizing a set of mini-batch normalization parameters from the one or more normalized feature maps; and
training a third layer to generate the prediction of an image class label for the training image based at least in part on the one or more normalized feature maps.

30. (canceled)
